(19) United States
(12) Patent Application Publication
(10) Pub. No.: US 2014/0328762 A1
(43) Pub. Date: Nov. 6, 2014

(54) Fluorogenic Peptide Probes and Assays

(71) Applicants: Yiyi Zhang, Atlanta, GA (US); Steven N. Dublin, Atlanta, GA (US); Gang Bao, Mabelton, GA (US)

(72) Inventors: Yiyi Zhang, Atlanta, GA (US); Steven N. Dublin, Atlanta, GA (US); Gang Bao, Mabelton, GA (US)

(21) Appl. No.: 14/110,002

(22) PCT Filed: Apr. 6, 2012
(86) PCT No.: PCT/US2012/032583

§ 371 Date: Apr. 10, 2014

Related U.S. Application Data
(60) Provisional application No. 61/472,368, filed on Apr. 6, 2011.

(51) Int. Cl. G01N 33/58; G01N 33/50

(52) U.S. Cl. CPC G01N 33/582; G01N 33/502; G01N 2500/10; G01N 2500/02
USPC 424/9.6; 530/324; 436/501; 435/7.1; 435/5

(57) ABSTRACT

Fluorogenic peptide probes specific for a target molecule are provided. Exemplary fluorogenic peptide probes include a peptide labeled with a fluorogenic label, wherein the fluorogenic peptide probe has an unstructured three dimensional conformation in the absence of the target molecule and a structured three dimensional conformation in the presence of the target polypeptide. The structured three dimensional conformation includes an alpha-helix that forms a coiled-coil with the target molecule allowing generation of a detectable signal from the fluorogenic label in response to an exciting amount of radiation.

### CROSS-REFERENCE TO RELATED APPLICATIONS

This application claims the benefit of priority of U.S. provisional application No. 61/472,368, filed Apr. 6, 2011, the content of it being hereby incorporated by reference in its entirety for all purposes.

### STATEMENT REGARDING FEDERALLY SPONSORED RESEARCH OR DEVELOPMENT

This invention was made with Government Support under Agreement PN2 EY018244-01 awarded by the National Institutes of Health. The Government has certain rights in the invention.

### FIELD OF THE INVENTION

The invention is generally directed to fluorogenic peptide probes and methods of their use.

### BACKGROUND OF THE INVENTION

Label-free biosensing of target molecules is a rapidly advancing technology with broad clinical and commercial potential. Biosensors often achieve impressive sensitivity and selectivity relative to many other sensing approaches (Oh, et al., Chem. Eur. J., 15:2244-2251 (2009)). For example, U.S. Pat. Nos. 7,399,591 and 7,081,336 to Bao et al. disclose dual resonance energy transfer molecular beacons that provide detectable signals for rapid, specific and sensitive hybridization determinations in viva.

Molecular beacons are oligonucleotides with one end modified with an attached quencher and the other end modified with an attached fluorophore. Molecular beacons rely on binding-specific conformational change to produce a detectable signal. In the absence of the target nucleic acid, the terminal ends of the molecular beacon anneal to each other bringing the fluorophore/quencher pair in proximity thereby minimizing fluorescence emission. Hybridization of the target nucleic acid to its complementary sequence in the middle of the molecular beacon causes a conformational change that separates the fluorophore/quencher pair resulting in a large increase in fluorescence emission.

Protein-based biosensors that rely on binding-specific conformational change to produce a detectable signal have also been developed (Oh, et al., Chem. Eur. J., 15:2244-2251 (2009)). Existing protein-based biosensors suffer from a low signal to noise ratio and solubility problems. Thus, there is a need for improved protein-based biosensors.

Therefore, it is an object of the invention to provide protein-based biosensors that have improved signal to noise ratios compared to existing technologies.

It is another object of the invention to provide methods of designing protein-based biosensors that can be used to detect molecules in living cells.

### SUMMARY OF THE INVENTION

Fluorogenic peptide probes (also referred to as peptide beacons) specific for a target molecule are provided. Exemplary fluorogenic peptide probes include a peptide labeled with a fluorogenic label, wherein the fluorogenic peptide probe has an unstructured three dimensional conformation in the absence of the target molecule and a structured three dimensional conformation in the presence of the target polypeptide. The structured three dimensional conformation includes an alpha-helix that forms a coiled-coil with the target molecule allowing generation of a detectable signal from the fluorogenic label in response to an exciting amount of radiation. In a preferred embodiment, the fluorogenic peptide probe contains two fluorogenic labels that form an H-dimer in the unstructured three-dimensional conformation. Suitable labels include, but are not limited to tetramethylrhodamine and rhodamine green.

Methods of detecting a target molecule are also provided. The methods include contacting a sample to be assayed for the presence of the target molecule with a fluorogenic peptide probe that specifically binds to or associates with the target molecule. Generation of a detectable signal is indicative of the presence of the target molecule. Typically, the target molecule is a protein such as motor protein, DNA-binding transcription factor, or viral capsid protein. Detecting the target can occur intracellularly or extracellularly.

Methods of screening for inhibitors of protein complex formation are also described. The methods include assaying a test compound for its ability to inhibit protein complex formation using the disclosed fluorogenic peptide probes to detect the presence of protein monomers. A detectable signal is indicative of monomers which is indicative that the test compound inhibits protein complex formation or polymerization. An example of protein complex assembly includes, but is not limited to viral particle assembly.

### BRIEF DESCRIPTION OF THE DRAWINGS

FIG. 1 is a schematic illustration of the peptide beacon design for detection of coiled coil-forming peptides. Sequences of coiled coils are characteristic of heptad repeats, designated (abcdefg)ₙ, wherein the a and d positions are usually occupied by hydrophobic residues that pack against and wrap around each other to form the hydrophobic core of an elongated left-handed super coil. The e and g positions are typically charged residues, participating in i to i′+5 electrostatic interactions. Residues at b, c and f positions are usually hydrophilic, and they are not directly involved in peptide-peptide interactions. The peptide beacon is composed of a recognition peptide, of which two cysteine residues are introduced at carefully selected b positions in the recognition peptide, and two attached 6-TAMRA fluorophores. In the absence of target molecules, flexibility of the recognition peptide and close proximity of the attached TAMRA fluorophores facilitate formation of intramolecular TAMRA H-dimer, which is characteristic of blue-shifted absorbance and substantially quenched fluorescence. The recognition peptide determines the target peptide binding specificity, while the TAMRA H-dimer signals the binding events. In the presence of target molecules, binding-induced conformational change of the recognition peptide into α helix dissociates the H-dimer, which in turn leads to significantly enhanced fluorescence readout. The coiled coil shown here is from the NMR structure of IAAL E3/K3 heterodimer (PDB ID 1U0I), and sidechains of isoleucines and leucines at a and d positions are illustrated to show packing between the K3 and E3 peptides.

FIGS. 2A and 2B show line graphs of absorbance versus wavelength (nm). FIG. 2A shows the absorbance spectra of the 225/244 beacon in the presence of AP-1 DNA and a negative control AT DNA. FIG. 2B shows the absorbance spectra of the GCN4 225/237 beacon in the presence of AP-1 DNA and a negative control. All samples contain 1 μM beacon, and concentration for DNA is 10 μM.

FIGS. 3A and 3B show line graphs of relative fluorescence intensity versus wavelength (nm) and FIG. 3C shows a line graph of relative fluorescence intensity versus concentration (nM). FIG. 3A shows the fluorescence intensity in the presence of AP-1 DNA and FIG. 3B shows intensity in the presence of AT DNA. Samples contain 100 nM GCN4 225/244 beacon and indicated concentration of DNA. FIG. 3A shows that the fluorescence intensity of GCN4 225/244 beacon is significantly enhanced by presence of increasing concentration of AP-1 DNA. FIG. 3B shows the negative control AT DNA does not impose any effects on the fluorescence intensity of GCN4 225/244 beacon. FIG. 3C shows the relationship between fluorescence intensity of GCN4 225/244 beacon at 580 nm and concentration of AP-1 DNA or AT DNA.

FIG. 4 is a line graph of absorbance versus wavelength (nm). The graph shows the absorbance spectra of 1 μM Rex beacon in the presence of 50 μM Rex RAN aptamer or TAR RNA.

FIGS. 5A and 5B show line graphs of relative fluorescence intensity versus wavelength (nm) and FIG. 5C shows a line graph of relative fluorescence intensity versus concentration (nM). Titration of fluorescence emission of Rex beacon with increasing concentration of Rex RNA aptamer or Tat TAR RNA. Samples contain 100 nM beacon is shown.

FIG. 6A is a schematic diagram of the peptide beacon in the presence and absence of the target protein. FIG. 6B is the structure of the DNA Ligase IV peptides. SEQ ID NOs: 1, 2, 3, 4, 5, and 6 are shown in order from top to bottom. FIG. 6C is a line graph of absorbance versus wavelength (nm) of probe and probe plus proteinase K. FIG. 6D is a bar graph of normalized fluorescence versus different LigIV beacons mixed with XRCC4. The C1 beacon yields highest fluorescence intensity, thus it is chosen for following studies. Concentration for all beacons is 2 μM, concentration of XRCC4 is 25

FIG. 7 is a line graph of absorbance versus wavelength (nm). The graph shows the absorbance spectra of C1 beacon. Beacon concentration is 1 μM, protein concentration is 40 μM. The A550/A520 ratio for beacon only, beacon with XRCC4 and beacon with truncated XRCC4 sample is 0.40, 0.46 and 0.38, respectively.

FIG. 8 is a line graph of relative fluorescence intensity versus concentration (μM). Fluorescence intensity of C1 beacon in the presence of increasing concentration of XRCC4 or truncated XRCC4 is shown. Concentration of C1 beacon is 2 μM.

FIG. 9 shows the sequences for the different JunLZ and FosLZ beacons. SEQ ID NOs:7-11 are shown from top to bottom, respectively.

FIG. 10 shows the structure of AP-1 complexed with a double-stranded DNA containing the AP-1 binding site.

FIGS. 11A-11H show line graphs of relative fluorescence intensity versus wavelength (nm). The graphs show a comparison of binding-induced fluorescence enhancement of JunLZ beacons. Concentration for all beacons is 1.0 and concentration of FosLZ or scrFosLZ is 10.0 μM. The assay was carried out in 0.1 M PBS, pH7.2 at room temperature. All longer beacons exhibit significantly enhanced fluorescence emission in the presence of FosLZ but not scrFosLZ (FIGS. 11A, C, E, and G). Shorter beacons show no or only slightly enhanced fluorescence emission in the presence of FosLZ (FIGS. 11B, D, F, H). Numbers above the curves represent signal to noise ratios calculated from the maxim emission values that centered around 585 nm. The Sb2b5 beacon shows abnormally high background fluorescence level, though, presence of either FosLZ or scrFosLZ leads to no alteration of its fluorescence intensity. The other 7 beacons exhibit background fluorescence levels ranging from 3000 to 6500. The JunLZ-Lb1b4 beacon shows highest signal to noise ratio.

FIGS. 12A and 12B show line graphs of absorbance versus wavelength (nm). The absorbance spectra of the JunLZ-Lb1b4 beacon in the presence of increasing concentration of FosLZ (FIG. 12A) or scrFosLZ (FIG. 12B) is shown. Note the gradual reversing of the major absorbance peak from 520 nm to 550 nm with increased concentration of FosLZ, which demonstrates dissociation of more and more TAMRA H-dimer. The negative control scrFosLZ does not impose any effects on the absorbance spectra, indicating that the interaction between JunLZ-Lb1b4 beacon and FosLZ is highly specific and indeed is due to formation of coiled coil leucine zipper.

FIGS. 13A and 13B show line graphs of relative fluorescence intensity versus wavelength (urn) and FIG. 13C shows a line graph of relative fluorescence intensity versus concentration (nM). Fluorescence enhancement of the JunLZ-Lb1b4 beacon is shown. The presence of increasing concentrations of FosLZ leads to gradually increased fluorescence intensity (FIG. 13A). The presence of increasing concentration of scrFosLZ has no effects (FIG. 13B). Note the gradual reversing of the major absorbance peak from 520 nm to 550 nm with increased concentration of FosLZ, which demonstrates dissociation of more and more TAMRA H-dimer. The negative control scrFosLZ does not impose any effects on the absorbance spectra, indicating that the interaction between JunLZ-Lb1b4 beacon and FosLZ is highly specific and indeed is due to formation of coiled coil leucine zipper.

FIGS. 14A and 14B show bar graphs of relative fluorescence intensity versus different peptide beacons. Non-specific fluorescence enhancement of peptide beacons by various unrelated coiled coil-forming peptides is shown. All reagents other than the beacons are loaded to 10 μM. The data represent relative fluorescence intensity at 580 nm (bandwidth 20 nm) collected by excitation at 520 nm (band width 15 nm). (FIG. 14A) All samples contain 100 nM K3-b1b3 beacon. Columns A, B, C, D, E are beacon only, beacon with unlabeled JunLZ-Lb1b4, beacon with unlabeled FosLZ, beacon with unlabeled GCN4 244, and beacon with E3 peptide, respectively. As compared with the beacon only sample, B, C, D, E shows fluorescence enhancement of 1.5-fold, 1.6-fold, 1.2-fold and 7.0-fold. (FIG. 14B) All samples contain 100 nM JunLZ-Lb1b4 beacon. Column A, B, C, D, E are beacon only, beacon with unlabeled K3-b1b3 peptide, beacon with E3 peptide, beacon with unlabeled GCN4 244, beacon with FosLZ. As compared with the beacon only sample, B, C, D, E shows fluorescence enhancement of 1.1-fold, 2.5-fold, 1.4-fold and 7.8-fold.

FIG. 15 shows the formation of an α-helical coiled-coil. (left) Helical wheel diagram of an amphipathic peptide sequence capable of participating in a coiled-coil assembly. Each peptide is composed of seven amino acid repeats termed heptads, in which hydrophobic and polar residues are spaced in an (i, i+4) arrangement. (right) Individual heptad-repeat peptides display unstructured random coil secondary structure in solution until they encounter a partner peptide. With this recognition event, a dramatic conformational change takes place and both peptides change their conformation in to a right-handed α-helix, and two strands are interwoven to form a heterodimeric left-handed coiled-coil.

FIG. 16A is a line graph of absorbance versus wavelength (nm). The graph indicates a shift in the wavelength between probe alone and probe plus proteinase k. FIG. 16B is a line graph of fluorescence versus wavelength (nm) showing an increase in fluorescence with probe plus proteinase k.

FIG. 17 shows peptide beacon designs and bar graph of S/N versus the different peptide beacons. SEQ ID NOs: 1-6 are shown.

### DETAILED DESCRIPTION OF THE INVENTION

### I. Fluorogenic Peptide Probes

A. Conformational Changes

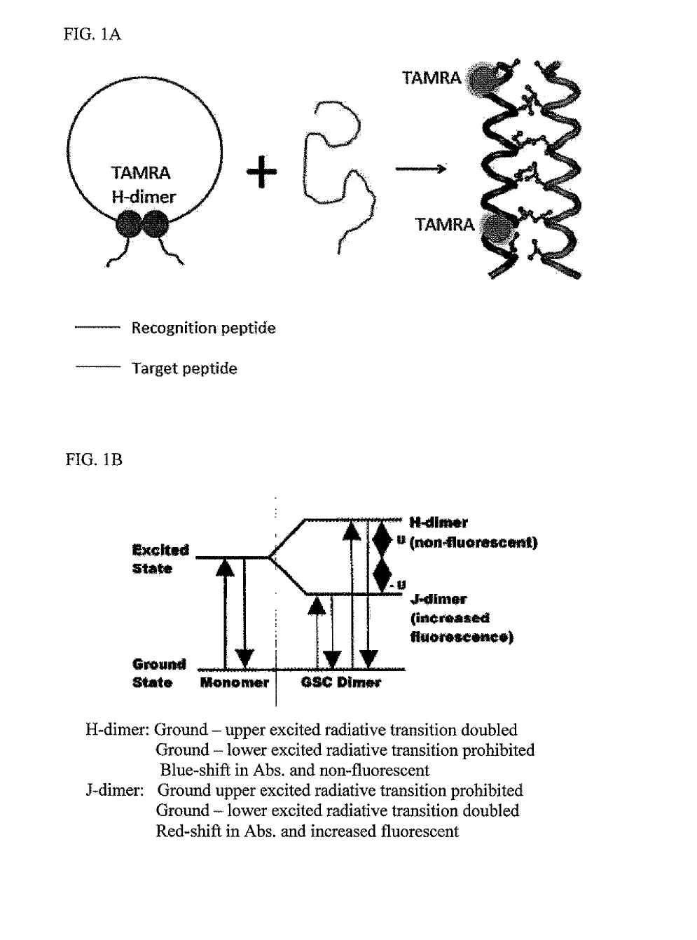

Fluorogenic peptide probes are described that have detectable labels attached to an unstructured and flexible peptide backbone. In one embodiment shown in FIG. 1, the fluorogenic peptide probes are formed by covalently attaching two fluorophores (TAMRA or Rhodamine Green) onto an unstructured and flexible peptide backbone. This allows the two fluorophore moieties to collide with each other and form a nonfluorescent dye H-dimer. When the non-fluorescent H-dimer is interrupted into two fluorophore monomers, the probes emit fluorescence signal that can be readily detected, for example by a microplate reader or a fluorescence microscope. In a preferred embodiment, the H-dimer is interrupted when the peptide probe undergoes a conformational change in the presence of its target molecule. The peptide probe transitions from an unstructured state into an α helix structure when the peptide associates with its target. The α helix of the peptide probe forms a coiled coil with the target allowing the labels to be detected. This special assembly is illustrated in FIG. 15.

The fluorogenic peptide probe can be utilized as an excitable probe to detect a target protein or nucleic acid in vivo or in vitro. The target protein can be an endogenous protein or a protein that has been genetically modified to display a fused peptide tag capable of forming an α helical coiled-coil with the peptide backbone of the probe.

B. Peptide Backbones

The peptide backbone of the fluorogenic peptide probes includes a sufficient number of amino acids to form an α helix. The amino acids can be naturally occurring amino acids or modified amino acids. Exemplary fluorogenic peptide probes (also referred to as peptide beacons) are provided in Table 1.

TABLE 1
Exemplary Peptide Beacons

| | |
|---|---|
| K3-b1b3 | K ICALKAK IAALKAK ICALKAGY (SEQ ID NO: 12) |
| K3-b1 | K ICALKAK IAALKAK IAALKAGY (SEQ ID NO: 13) |
| E3 | E IAALEKE IAALEKE IAALEKGY (SEQ ID NO: 14) |
| scrE3 | E AAAAEKE AAAAEKE AAAAEKGY (SEQ ID NO: 15) |
| *JunLZ-Sb1b3 | VCELEER VKTLKAQ ICELKST RNMLREQ VAQLA |
| | (SEQ ID NO: 16) |
| *JunLZ-Sb3b5 | VAELEER VKTLKAQ ICELKST RNMLREQ VCQLA |
| | (SEQ ID NO: 17) |
| *JunLZ-Sb1b4 | VCELEER VKTLKAQ ISELKST RCMLREQ VAQLA |
| | (SEQ ID NO: 18) |
| *JunLZ-Sb2b5 | VAELEER VCTLKAQ ISELKST RNMLREQ VCQLA |
| | (SEQ ID NO: 19) |
| *JunLZ-Lb1b3 | VCELEER VKTLKAQ ICELKST RNMLREQ VAQLKQK |
| | VA (SEQ ID NO: 20) |
| *JunLZ-Lb3b5 | VAELEER VKTLKAQ ICELKST RNMLREQ VCQLKQK |
| | VA (SEQ ID NO: 21) |
| *JunLZ-Lb1b4 | VCELEER VKTLKAQ ISELKST RCMLREQ VAQLKQK |
| | VA (SEQ ID NO: 22) |
| *JunLZ-Lb2b5 | VAELEER VCTLKAQ ISELKST RNMLREQ VCQLKQK |
| | VA (SEQ ID NO: 23) |
| *FosLZ | TDTLQAE TDQLEDE KSALQTE IANLLKE KEKLEFI LA |
| | (SEQ ID NO: 24) |
| *scrFosLZ | TDTAQAE TDQAEDE KSAAQTE IANALKE KEKAEFI LA |
| | (SEQ ID NO: 25) |
| LigIV-C1 | CREYDSYGDSYFIDTDLNQLKEVFSGIKCG (SEQ ID NO: 2) |
| LigIV-C16 | AREYDSYGDSYFIDTCLNQLKEVFSGIKCG (SEQ ID NO: 3) |
| LigIV-N | CGYDSYGDSYFIDTDLNQLKEVFGC (SEQ ID NO: 5) |
| LigIV-C | CGDTDLNQLKEVFSGIKNSNEQGC (SEQ ID NO: 6) |
| *GCN4-237 | CDPAALKRARNTCAARRSRARKLQR MKQLEDK |
| | VEELLSK NYHLENE VARLKKL VGER (SEQ ID NO: 26) |
| *GCN4-244 | CDPAALKRARNTEAARRSRCRKLQR MKQLEDK |
| | VEELLSK NYHLENE VARLKKL VGER (SEQ ID NO: 27) |
| REX-ARM | MPCTRRRPRRSQRCRPGY (SEQ ID NO: 28) |

*These peptides are expressed as recombinant proteins, fused at the C-end of maltose binding protein. A flexible linker, GGSGG is inserted at the fusion site. The amino acids are in single letter code.

The synthetic peptides are acetylated and aminated at their N- and C-termini, respectively. Peptides involved in formation of α helical coiled coils or leucine zippers are listed as heptad repeats. In one embodiment, the peptide beacons were prepared by conjugating TAMRA 6-maleimide to the —SH groups of the cysteine residues in bold.

The K3 peptides are modified from the original sequence of K IAALKEK IAALKEK IAALKE (SEQ ID NO:29). In addition to introduced cysteines residues as indicated, the glutamic acids atf positions of the heptad repeats were replaced with alanines to enhance solubility of the K3-b1b3 beacon.

The JunLZ peptides are based on JunW_{Ph2}, a variant cJun leucine zipper peptide selected from partially randomized phage display library against the leucine zipper of cFos. The Sb1b3, Sb3b5, Sb1b4 and Sb2b5 sequences are directly modified from JunW_{Ph2}. The Lb1b3, Lb3b5, Lb1b4 and Lb2b5 sequences are identical to Sb1b3, Sb3b5, Sb1b4 and Sb2b5, respectively, except that they are extended with four additional residues (KQKV) from native cJun right after the leucine zipper. S means shorter, L means longer. All cysteine residues are introduced to b positions of indicated heptad repeats.

The LigIV peptides are based on amino acids 755-782 of human DNA ligase IV, a region responsible for binding to XRCC4 dimer. GCN4 peptides are based on the bZip domain of GCN4 protein (amino acids 226-281). An additional cysteine residue was introduced at its N-end. E237 or A244 are mutated to cysteine for GCN4-237 and GCN4-244, respectively. Both mutations are within the basic region responsible for DNA binding. Rex-ARM is modified from the original arginine rich motif of Rex (Rex-ARM), sequence of which is MPKTRRRPRRSQRKRP (SEQ ID NO:30).

C. Labels

Fluorogenic peptide probes can be labeled with a wide variety of markers or labels. The markers can include, but are not limited to, fluorophores, dyes, nanoparticles, quantum dots, or a combination thereof. Preferably the label forms an H-dimer when the probe is in its unstructured conformation. Exemplary fluorophores include, but are not limited to tetramethylrhodamine (TMR), TAMRA or Rhodamine Green.

### III. Methods of Using Fluorogenic Peptide Probes

A. Methods of Detecting Target Proteins

The fluorogenic peptide probes are useful for detecting target molecules including proteins and nucleic acids. The target proteins can be native proteins or can be modified proteins.

1. Detecting Native Proteins

The methods for detecting a target molecule include combining the fluorogenic peptide probe with a sample to be assayed for the presence of the target molecule. The sample can be a biological sample obtained from a human subject including but not limited to a fluid, tissue, or cells. The fluorogenic peptide probe is designed to specifically bind or specifically associate with an amino acid sequence or a specific conformation of the target protein. In some embodiments, the fluorogenic peptide probe forms an α-helix in the presence of the target protein. The α-helix of the fluorogenic peptide probe forms a coiled-coil complex with the target protein allowing the label to be detected.

2. Detecting Modified Proteins

Modified proteins can be detected with the disclosed fluorogenic peptide probes. The modified proteins can be any protein that has been altered to contain a specific peptide sequence tag wherein the tag binds to the fluorogenic peptide probe. The modified protein to be detected can be added to a sample or can be expressed as a recombinant protein by a cell or organism.

B. Methods of Screening

The fluorogenic peptide probes can also be used to screen for compounds that inhibit polymerization or complex formation between monomeric units. Typically, the probes are used to screen for small molecules that inhibit the ability of proteins to form complexes. In one embodiment, the fluorogenic peptide probes are used to screen for drugs that inhibit viral capsid formation or inhibit viral particle assembly.

Inhibition of viral particle assembly can be determined in vitro by adding a test compound to a sample containing virus particle monomers and a fluorogenic peptide probe specific for the virus particle monomers. A detectable amount of signal relative to a control is indicative that the test compound inhibits viral particle formation.

Another method includes administering a test compound to a cell infected with a virus and containing a fluorogenic peptide probe. The method also includes exposing the cell to an exciting amount of radiation, wherein a detectable signal relative to a non-infected cell indicates that the test compound is an inhibitor of viral capsid polymerization. The virus is typically a retrovirus such as HIV or hepatitis.

Another embodiment provides a method of screening for an inhibitor of protein complex formation by administering a test compound to a sample containing at least two proteins that form a complex. A detectable signal relative to a control cell indicates that the test compound is an inhibitor of a protein complex formed by the two proteins.

### EXAMPLES

### Methods and Materials

1. Reagents.

All synthetic peptides are from GenScript. TAMRA-6-maleimide is from InVitrogen. Amylose agarose resin is from New England Biolabs. Ni-NTA agarose and proteinase K (>600 mAU/ml) are from Qiagen. Coomassie Plus protein assay reagent and Zeba spin desalting columns are from Pierce.

2. Concentration Determination of Peptides and Proteins.

Concentrations of synthetic peptides are determined by measuring the absorbance of peptide solutions at 275 nm, assuming a molar extinction coefficient of 1400 M⁻¹cm⁻¹ for tyrosine. Concentrations of proteins are determined with Coomassie Plus protein assay reagent following manufacturer's instruction.

3. Plasmid Construction.

a. pMBP

This plasmid was used to express the recombinant MBP protein in E. coli. The pMal-C4X plasmid (New England Biolabs) was linearized with EcoRI. The generated cohesive ends were blunted by incubating with Pfu DNA polymerase in the presence of four dNTPs. The as-prepared DNA was circularized by self-ligation to give pMBP plasmid.

b. pMBP-E3

This plasmid was used to express the recombinant fusion protein of maltose binding protein and E3 peptide (MBP-E3) in E. coli. The coding sequence of E3 peptide was obtained by hybridize 5′-GATCCGGAGGTAGTGGTGGAGAGATCGCTGCACTGGAGAAAGAAAT TG CAGCTCTCGAAAAGGAGATCGCTGCTCTGGAGAAGCCATAAG-3′ (SEQ ID NO:31) with 5′-TCGACTT ATGGCTTCTCCAGAGCAGCGATCTCCTMCGAGAGCTGCAATTTCTT TCTCCAGTGC AGCGATCTCTCCACCACTACCTCCG-3′ (SEQ ID NO:32). The resulting double-stranded DNA was inserted into the pMal-C4X vector (New England Biolabs) that has been digested with BamHI and SalI. The obtained plasmid was named pMBP-E3.

c. pQE-MBP

This plasmid was used as the vector to construct plasmids for expression in E. coli of recombinant fusion proteins of MBP and Jun leucine zipper, Fos leucine zippers as wells as GCN4 peptides. The MBP coding sequence was amplified from pMal-C4X with 5′-GTTACTGG ATCCAAAATCGAAGAAGGTAAACTGGTAATCTGG-3′ (SEQ ID NO:33) and 5′-GTTACTGTCGACAGTC TGCGCGTCTTTCAGGGCTTC-3′ (SEQ ID NO:34). The PCR product was digested with BamHI and SalI and then inserted into pQE-80L plasmid (Qiagen) that had been linearized with same restriction enzymes. The obtained plasmid was named pQE-MBP.

d. pQE-MBP-JunLZ Plasmids

These plasmids were used to express the fusion proteins of MBP and various Jun leucine zipper peptides in E. coli for preparation of JunLZ beacons. The plasmids were constructed by ligating pQE-MBP vector that had been digested with SalI and PstI with various oligo DNA duplexes.

b1C duplex was formed by hybridizing following oligos:

b1C sense:
(SEQ ID NO: 35)
5′-TCGACGGCGGCAGCGGCGGCGTGTGCGAACTGGAAGAACGCGTGAA-
3′
b1C antisense:
(SEQ ID NO: 36)
5′-GGTTTTCACGCGTTCTTCCAGTTCGCACACGCCGCCGCTGCCGCCG-
3′

b1A duplex was formed by hybridizing following oligos:

b1A sense:
(SEQ ID NO: 37)
5′-TCGACGGCGGCAGCGGCGGCGTGGCAGAACTGGAAGAACGCGT
GAA-3′
b1A antisense:
(SEQ ID NO: 38)
5′-GGTTTTCACGCGTTCTTCCAGTTCTGCCACGCCGCCGCTGCCG
CCG-3′

b2C duplex was formed by hybridizing following oligos:

b2C sense:
(SEQ ID NO: 39)
5′-TCGACGGCGGCAGCGGCGGCGTGGCAGAACTGGAAGAACGCG
TGTG-3′
b2C antisense:
(SEQ ID NO: 40)
5′-GGTGCACACGCGTTCTTCCAGTTCTGCCACGCCGCCGCTGCC
GCCG-3′

b3C duplex was formed by hybridizing following oligos:

b3C sense:
(SEQ ID NO: 41)
5′-AACCCTGAAAGCGCAGATTTGCGAACTGAAAAGCACCCGCAACATG
C-3′
b3C antisense:
(SEQ ID NO: 42)
5′-CGCAGCATGTTGCGGGTGCTTTTCAGTTCGCAAATCTGCGCTTTCAG-
3′

b4C duplex was formed by hybridizing following oligos:

b4C sense:
(SEQ ID NO: 43)
5′-AACCCTGAAAGCGCAGATTAGCGAACTGAAAAGCACCCGCTGCATG
C-3′
b4C antisense:
(SEQ ID NO: 44)
5′-CGCAGCATGCAGCGGGTGCTTTTCAGTTCGCTAATCTGCGCTTTCAG-
3′

b4N duplex was formed by hybridizing following oligos:

b4N sense:
(SEQ ID NO: 45)
5′-CACCCTGAAAGCGCAGATTAGCGAACTGAAAAGCACCCGCAACATG
C-3′
b4N antisense:
(SEQ ID NO: 46)
5′-CGCAGCATGTTGCGGGTGCTTTTCAGTTCGCTAATCTGCGCTTTCAG-
3′

Sb5C duplex was formed by hybridizing following oligos:

Sb5C sense:
(SEQ ID NO: 47)
5′-TGCGCGAACAGGTGTGCCAGCTGGCGTAACTGCA-3′
Sb5C antisense:
(SEQ ID NO: 48)
5′-GTTACGCCAGCTGGCACACCTGTTCG-3′

Sb5A duplex was formed by hybridizing following oligos:

Sb5A sense:
(SEQ ID NO: 49)
5′-TGCGCGAACAGGTGGCACAGCTGGCGTAACTGCA-3′
Sb5A antisense:
(SEQ ID NO: 50)
5′-GTTACGCCAGCTGTGCCACCTGTTCG-3′

Lb5C duplex was formed by hybridizing following oligos:

Lb5C sense:
(SEQ ID NO: 51)
5′-TGCGCGAACAGGTGTGCCAGCTGAAACAGAAAGTGGCGTAACTGCA-
3′
Lb5C antisense:
(SEQ ID NO: 52)
5′-GTTACGCCACTTTCTGTTTCAGCTGGCACACCTGTTCG-3′

Lb5A duplex was formed by hybridizing following oligos:

Lb5A sense:
(SEQ ID NO: 53)
5′-TGCGCGAACAGGTGGCACAGCTGAAACAGAAAGTGGCGTAACTGCA-
3′
Lb5A antisense:
(SEQ ID NO: 54)
5′-GTTACGCCACTTTCTGTTTCAGCTGTGCCACCTGTTCG-3′

Ligate pQE-MBP SalI-PstI vector with:

b1C duplex, b3C duplex, and Sb5A duplex to get pQE-MBP-JunLZ-Sb1b3;
b1C duplex, b4C duplex, and Sb5A duplex to get pQE-MBP-JunLZ-Sb1b4;
b2C duplex, b4N duplex, and Sb5C duplex to get pQE-MBP-JunLZ-Sb2b5;
b1A duplex, b3C duplex, and Sb5C duplex to get pQE-MBP-JunLZ-Sb3b5;
b1C duplex, b3C duplex, and Lb5A duplex to get pQE-MBP-JunLZ-Lb1b3;
b1C duplex, b4C duplex, and Lb5A duplex to get pQE-MBP-JunLZ-Lb1b4;
b2C duplex, b4N duplex, and Lb5C duplex to get pQE-MBP-JunLZ-Lb2b5;
b1A duplex, b3C duplex, and Lb5C duplex to get pQE-MBP-JunLZ-Lb3b5.

e. pQE-MBP-FosLZ

This plasmid was used to express the fusion protein of MBP and the cFos leucine zipper (MBP-FosLZ) in E. coli. The plasmid was constructed by ligating pQE-MBP vector that had been digested with SalI and PstI with FosLZ duplex 1, 2, and 3.

FosLZ duplex 1 was formed by hybridizing following oligos:

(SEQ ID NO: 55)
1 sense: 5′-
TCGACGGCGGCAGCGGCGGCACCGATACCCTGCAAGCGGAAACC-3′
(SEQ ID NO: 56)
1 antisense: 5′-
GATCGGTTTCCGCTTGCAGGGTATCGGTGCCGCCGCTGCCGCCG-3′

FosLZ duplex 2 was formed by hybridizing following oligos:

(SEQ ID NO: 57)
2 sense: 5′-
GATCAGCTGGAAGATGAAAAAAGCGCGCTGCAAACCGAAATTGCGA
A-3
(SEQ ID NO: 58)
2 antisense: 5′-
CAGGTTCGCAATTTCGGTTTGCAGCGCGCTTTTTTCATCTTCCAGCT-3′

FosLZ duplex 3 was formed by hybridizing following oligos:

(SEQ ID NO: 59)
3 sense: 5′-
CCTGCTGAAAGAAAAAGAAAAACTGGAATTTATTCTGGCGTAACTGC
A-3′
(SEQ ID NO: 60)
3 antisense: 5′-
GTTACGCCAGAATAAATTCCAGTTTTTCTTTTTCTTTCAG-3′

f. pQE-MBP-scrFosLZ

This plasmid was used to express the fusion protein of MBP and the scrambled cFos leucine zipper (MBP-scrFosLZ) in E. coli. The plasmid was constructed by ligating pQE-MBP vector that had been digested with SalI and PstI with scrFosLZ duplex 1, 2, and 3.

scrFosLZ duplex 1 was formed by hybridizing following oligos:

(SEQ ID NO: 61)
scrFos 1 sense: 5′-
TCGACGGCGGCAGCGGCGGCACCGATACCGCGCAGGCGGAAACC-3′
(SEQ ID NO: 62)
scrFos 1 antisense: 5′-
GATCGGTTTCCGCCTGCGCGGTATCGGTGCCGCCGCTGCCGCCG-3′

scrFosLZ duplex 2 was formed by hybridizing following oligos:

(SEQ ID NO: 63)
scrFos 2 sense: 5′-
GATCAGGCGGAGGATGAAAAAAGCGCGGCGCAGACCGAAATTGCG-
3′
(SEQ ID NO: 64)
scrFos 2 antisense: 5′-
CGTTCGCAATTTCGGTCTGCGCCGCGCTTTTTTCATCCTCCGCCT-3′

scrFosLZ duplex 1 was formed by hybridizing following oligos:

(SEQ ID NO: 65)
scrFos 3 sense: 5′-
AACGCGCTGAAAGAAAAAGAAAAAGCGGAATTTATTCTGGCGTAAC
TGCA-3′
(SEQ ID NO: 66)
scrFos 3 antisense: 5′-
GTTACGCCAGAATAAATTCCGCTTTTTCTTTTTCTTTCAGCG-3′

g. pQE-MBP-GCN4/237 and pQE-MBP-GCN4/244 Plasmids

These plasmids were used for expression in E. coli recombinant proteins of MBP and GCN4 bZip peptides for preparation of GCN4-237 and GCN4-244 beacons. These plasmids were constructed by ligating pQE-MBP vector that has been linearized with SalI and PstI with various duplex oligo DNAs.

GCN4 duplex 1 was formed by hybridizing following oligos:

(SEQ ID NO: 67)
GCN4 1 sense: 5′-
TCGACGGCGGCAGCGGCGGCTGCGATCCGGCGGCGCTGAAACGCGC
GC-3′
(SEQ ID NO: 68)
GCN4 1 antisense: 5′-
TTGCGCGCGCGTTTCAGCGCCGCCGGATCGCAGCCGCCGCTGCCGCC
G-3′

GCN4 duplex 2 was formed by hybridizing following oligos:

(SEQ ID NO: 69)
GCN4 2 sense: 5′-
GCAACACCTGCGCGGCGCGCCGCAGCCGCGCGCGCAAACTGCAACG-
3′
(SEQ ID NO: 70)
GCN4 2 antisense: 5′-
CATGCGTTGCAGTTTGCGCGCGCGGCTGCGGCGCGCCGCGCAGGTG-
3′

GCN4 duplex II was formed by hybridizing following oligos:

(SEQ ID NO: 71)
GCN4 II sense: 5′-
GCAACACCGAAGCGGCGCGCCGCAGCCGCTGCCGCAAACTGCAACG-
3′
(SEQ ID NO: 72)
GCN4 II antisense: 5′-
CATGCGTTGCAGTTTGCGGCAGCGGCTGCGGCGCGCCGCTTCGGTG-3′

GCN4 duplex 3 was formed by hybridizing following oligos:

(SEQ ID NO: 73)
GCN4 3 sense: 5′-
CATGAAACAGCTGGAAGATAAAGTGGAAGAACTGCTGAGCAAAAAC
TATC-3′
(SEQ ID NO: 74)
GCN4 3 antisense: 5′-
AGATGATAGTTTTTGCTCAGCAGTTCTTCCACTTTATCTTCCAGCTGT
TT-3′

GCN4 duplex 4 was formed by hybridizing following oligos:

(SEQ ID NO: 75)
GCN4 4 sense: 5′-
ATCTGGAAAACGAAGTGGCGCGCCTGAAAAAACTGGTGGGCGAACG
CTAACTGCA-3′
(SEQ ID NO: 76)
GCN4 4 antisense: 5′-
GTTAGCGTTCGCCCACCAGTTTTTTCAGGCGCGCCACTTCGTTTTCC-3′

Ligate pQE-MBP SalI-PstI vector with:

duplex 1, duplex 2, duplex 3 and duplex 4 to get pQE-MBP-GCN4/237
duplex 1, duplex II, duplex 3 and duplex 4 to get pQE-MBP-GCN4/237

h. pQE-XRCC4

This plasmid was used to express full length human XRCC4 protein in E. coli. The XRCC4 coding sequence was amplified from pENTR-XRCC4 (a kind gift from Dr. William Dynan at Georgia Health Sciences University) with 5′-GTTATTGGATCCGAGAGAAAAATA AGCAGAATCCACCTTGTTTC-3′ (SEQ ID NO: 77) and 5′-GTTATTGTCGACTTAAATCTCATCAAAGAGG TCTTCTGGGCTG-3′ (SEQ ID NO:78). The PCR product was digested with BamHI and SalI, and inserted into pQE-80L plasmid that had been cleaved with same enzymes. The obtained plasmid was named pQE-XRCC4.

i. pQE-XRCC4-trunc

This plasmid was used for expression in E. coli the truncated XRCC4 protein that does not have DNA ligase IV binding site. Coding sequence of amino acids 2-150 was amplified from pENTR-XRCC4 with 5′-CGTAGTACTGGATCCGAGAGAAAAATAAG CAGAATCCACCTT GTTTC-3′ (SEQ ID NO:79) and 5′-TGCATGAGCTCCCTTTCATTTTCT TTCTGCAGG TGCTCATTTTTGG-3′ (SEQ ID NO:80). The PCR product was digested with BamHI and SacI, and ligated into pQE-80L vector that had been digested with same enzymes. The obtained plasmid was named pQE-XRCC4-trunc.

j. pTag-XRCC4.

This plasmid was used for expression in U2OS cells of full length human XRCC4 protein as the target for testing live cell protein imaging with LigIV-C1 beacon. The XRCC4 coding sequence was amplified with primers 5% GCCCTGGGATCCGCCACCATGGA GAGAAAAATAAGCAGAATCCACCTTGTTTCT-3′ (SEQ ID NO:81) and 5′-GCCATGTCTAGATTAAATCT CATCAAAGAGGTCTTCTGGGCTGC-3′ (SEQ ID NO:82). The PCR product was digested with BamHI and XbaI and ligated into mammalian expression vector pTagGFP-N (Evrogen) that had been digested with same enzymes. The resulted plasmid was named pTag-XRCC4.

k. pTag-XRCC4-trunc

This plasmid was used for expression in U2OS cells the truncated XRCC4 protein that does not have DNA ligase IV binding site. The protein was used as a negative control for testing live cell protein imaging with LigIV-C1 beacon. The coding sequence of amino acids 1-150 of human XRCC4 was amplified from pENTR-XRCC4 with 5′-GCCCTGGG ATCCGCCACCATGGAGAGAAAAATAAGCAGAATCCACCTTGTTTCT-3′ (SEQ ID NO:83) and 5′-TGCAT TCTAGACTACCTTTCATTTTCTTTCTGCAGGTGCTCATTTTTGGCTTGA TTTTCTGCAA TGGTGTCCAAGCA-3′ (SEQ ID NO:84). The PCR product was digested with BamHI and XbaI and ligated into mammalian expression pTagGFP-N vector that had been digested with same enzymes. The resulted plasmid was named pTag-XRCC4-trunc.

4. Protein Expression and Purification.

Recombinant proteins were expressed at 37° C. in BL21(DE3) cells. Expression is initiated by adding IPTG to final concentration of 1 mM when O.D. 600 of the cultures reaches 0.8. Cells are grown another 4-5 hours before being harvested. Recombinant MBP-fusion proteins were purified with an amylase affinity column, following manufacturer's instructions. Recombinant proteins that are not fused with MBP were purified by Ni-NTA chromatography, following manufacturer's instruction. Purified proteins are stored at −80° C. in 0.1 M PBS, pH 7.2.

5. Preparation and Purification of Peptide Beacons.

Synthetic peptide solution of 50 μM is freshly prepared by dissolving lyophilized peptide powder in 20 mM phosphate buffer, pH 7.4. To this solution, freshly prepared stock solution of TAMRA-6-maleimide in dimethylformamide (DMF) is added to final concentration of 200 μM. The mixture is incubated at room temperature for 1 hour. To purify peptide beacons from excess free dyes, the reaction mixture is loaded onto a C18 reversed phase column (Restek) and eluted with a linear gradient of 10-60% acetonitrile (within 30 min) in H₂O with 0.1% TFA (4.8 ml/min). The purification process is monitored by measuring absorbance at 550 nm. The peak containing peptide beacons is pooled into a glass vial, and vacuumed to remove acetonitrile and TFA. The pH of the remaining solution is adjusted to 7.2 with 1 M Na₂HPO₄. After this, the beacon solution is aliquoted, snap-frozen with liquid nitrogen, and stored at −80° C. for later use. For labeling of peptides fused to the C-end of maltose binding protein, the fusion protein is first reduced with 5 mM DTT in 0.1 M PBS (pH 7.2) by incubating at room temperature for 1-2 hours. Excess DTT is removed by passing the protein solution through a Zeba spin desalting column pre-equilibrated with 0.1 M PBS and 5 mM EDTA. The concentration of the fusion protein is then adjusted to 50 μM, and TAMRA-6-maleimide is added to final concentration of 200 μM. The mixture is incubated at room temperature for 1 hour, after which the mixture is treated again with a Zeba spin desalting column pre-equilibrated with 0.1 M PBS to remove excess free dyes. After this, the labeled protein (beacon) is aliquoted, snap-frozen with liquid nitrogen, and stored at −80° C. for later use.

6. Determination of Peptide Beacon Concentration.

Five microliters of proteinase K was added into 95 μl diluted beacons (in 0.1M PBS). Digestion was allowed for 10 minutes at 37° C. Preliminary experiments show that this condition results in complete digestion of peptide beacons, and longer incubation time does not lead to further increase of the absorbance at 550 nm, the major absorbance peak of monomeric TAMRA fluorophore. The absorbance at 550 nm was used to quantitate the concentration of TAMRA-6 in solution, assuming the molar extinction coefficient at 550 nm to be 95000 M⁻¹cm⁻¹. Beacon concentration is half of the TMR concentration multiplied by dilution folds.

7. Absorbance and Emission Spectra of Peptide Beacons.

All spectra data were obtained with Safire II microplate reader (TECAN). Data was acquired right after mixing the beacon with its target. Longer incubation results in no further increase in signal intensity.

8. Live Cell Imaging of XRCC4 Protein with the LigIV-C1 Peptide Beacon.

U2OS cells were transfected with either pTag-XRCC4 or pTag-XRCC4-trunc by electroporation, together with pTag-GFP. Cells were allowed to grow for 24 hours before being imaged. For live cell imaging of XRCC4, 4 μM of LigIV-C1 peptide beacon in 10 mM PBS was microinjected into cell nuclei of U2OS cells that exhibit positive GFP fluorescence. Beacon fluorescence images were taken right after injection, by using standard TRITC filter set.

### Example 1

### K3 Beacon Technology

K3 and E3 are a pair of artificially designed peptides. Both peptides are non-structured individually, however, when mixed together, they interact to form a coiled-coil. It is this conformational change that opens the H-dimer of tetramethylrhodamine (TAMRA), as illustrated in FIG. 1.

To make the K3 peptide appropriate for beacon preparation, the original K3 peptide (K IAALKEK IAALKEK IAALKE) was modified by introducing two cysteine residues at b positions of the first (b1) and third heptads (b3). The modified peptide is named K3-b1b3, which has a sequence of K ICALKAK IAALKAK ICALKA GY (SEQ ID NO:12). The tyrosine residue (Y) was added for the purpose of peptide concentration determination. The glycine residue was added to separate the tyrosine residue from the other part of the peptide, in case the huge side-chain of tyrosine may interfere with the coiled-coil formation.

As seen with the original K3 peptide, the K3-b1b3 peptide is non-structured, and the addition of E3 peptide induces formation of heterodimeric coiled-coil (data not shown). Meanwhile, a scrambled E3 peptide, wherein all the hydrophobic residues (I and L) at a and d positions are replaced with alanines, does not induce formation of coiled-coil (data not shown). So, the interaction between K3-b1b3 and E3 peptide is highly specific.

With cysteine residues in the K3-b1b3 peptide, two 6-TAMRA fluorophores are readily incorporated via the maleimide chemistry. Close proximity of two TAMRA fluorophores in the same peptide facilitates formation of H-dimer (a well-known phenomenon for xanthene fluorophores including rhodamines), which is expected to change the conformation of the free K3-b1b3 peptide to a more confined one (or a loop). A significant difference between the CD spectra of the free K3-b1b3 peptide and the TAMRA labeled K3-b1b3 was observed (data not shown).

More importantly, addition of equimolar E3 peptide to the K3-b1b3 beacon induces formation of a helical coiled coil, while addition of the scrE3 peptide does not (data not shown). Accompanying this conformational change, optical properties of the beacon alter as well. The beacon itself exhibits maxim absorbance at 520 nm, 30 nm blue-shifted from the absorbance peak of monomeric 6-TAMRA, indicating formation of TAMRA H-dimer (data not shown). The H-dimer must be an intramolecular one, since a single-labeled peptide (K3-b1) at 2-fold concentration exhibits maxim absorbance at 550 nm (data not shown), same as monomeric TAMRA fluorophore. In addition, the fluorescence intensity of K3-b1 b3 beacon increases linearly with concentration (data not shown). Addition of the E3 but not the scrE3 peptide to the K3-b1b3 beacon results in reversing of the major absorbance peak from 520 nm to 550 nm, indicating specific binding-induced dissociation of the TAMRA H-dimer (data not shown).

Corresponding to the absorbance change, fluorescence emission of the K3-b1b3 beacon is also significantly enhanced by the presence of the E3 but not the scrE3 peptide (data not shown). Titration of 1 μM K3-b1b3 beacon results in a maximum fluorescence enhancement of about 12-fold. Curve fitting result indicates that the K_{d} value for binding between K3-b1b3 beacon and E3 peptide is 766 nM.

Collectively, the above-described data demonstrate that specific binding between the K3-b1b3 beacon and the E3 peptide induces conformational change of the beacon, which in turn dissociates the non-fluorescent TAMRA H-dimer and thus greatly facilitates fluorescence emission.

To further characterize the K3-b1b3 beacon, titration of lower concentration (10 nM) of K3-b1b3 beacon has also been performed. Slightly lower fluorescence enhancement was observed at the highest tested concentration (data not shown). The K3-b1b3 beacon functions in a very wide concentration range from nM to μM. Curve fitting gives a K_{d} value of 738 nM, which agrees very well with the result of 1 μM beacon titration (766 nm).

As reported in the literature, the K_{d} value for binding between the original K3 and E3 peptides is 70 nm, which is one order lower as compared with the K_{d} value for binding between the K3-b1b3 beacon and the E3 peptide. The difference should come from the energy bather imposed by the TAMRA H-dimer. Since the sequence of K3-b1b3 peptide has been modified from that of the original K3 peptide, the K_{d} value for binding between unlabeled K3-b1b3 and E3 peptides was re-measured by CD spectra analysis (data not shown). Curve fitting gives a K_{d} value of 45 nM. This is a reasonable result, since the three glutamic acids at f positions were replaced with alanines, and that alanine has higher a helix propensity than glutamic acid. Comparing with the K_{d} value for binding between the K3-b1b3 beacon and the E3 peptide, ˜17-fold decrease of binding affinity is imposed by the TAMRA H-dimer. Previous studies reported a 3-fold decrease of binding affinity for a heterodimeric H-dimer of fluorescein and TAMRA. Given that fluorescein needs a much higher concentration (˜100 fold) as compared with TAMRA to form H-dimer, the heterodimer should impose lower energy barrier.

The application of using the K3-b1b3 beacon for detection of E3-fusion proteins has also been tested. For this purpose, a recombinant maltose binding protein (MBP) with E3 fused at its C-terminal end was created. Similar amount of fluorescence enhancement was observed, as compared with the free E3 peptide (data not shown). The wild-type MBP does not have effects on fluorescence emission of the K3-b1b3 beacon (data not shown). Specific binding between K3-b1b3 beacon and MBP-E3 has also been confirmed by gel-shift assay (data not shown).

In summary, the K3/E3 peptides have been employed as a model system to test the feasibility of using TAMRA H-dimer-based peptide beacon for biomolecular detection. Of note, the conformation of the K3-b1b3 beacon after binding with E3 is a helix, this secondary structure of the probe has not been reported for any earlier peptide beacon study.

Other probes include the following:

(SEQ ID NO: 85)

| | |
|---|---|
| K3 1 nm: | Ac- K ICALKEK ICALKEK IAALKE GWP -NH2 |

(SEQ ID NO: 86)

| | |
|---|---|
| K3 2 nm: | Ac- K ICALKEK IAALKEK ICALKE GWP -NH2 |

(SEQ ID NO: 87)

| | |
|---|---|
| E3 coil: | Ac- E IAALEKE IAALEKE IAALEK GWP -NH2 |

### Example 2

### GCN4 244 Beacon and Rex Beacon

The GCN4 244 beacon and Rex beacon were developed to generalize the applications of TAMRA H-dimer-based peptide beacons for nucleic acids detection.

GCN4 is the yeast homolog of mammalian AP-1 transcription factor, though, instead of being heterodimeric, GCN4 functions as a homodimer. GCN4 is also a bZIP protein, and its dimerization is mediated by coiled coil formation. To develop the beacon that detects double-stranded DNA, the basic region of bZIP domain of GCN4 was mutated to bear 2 cysteine residues, which are used for conjugation of TAMRA fluorophores. It has been previously reported that, in the absence of target DNA, the leucine zipper of GCN4 can dimerize, though, the basic region remains non-structured. Upon complexation with a target DNA, the basic region changes its conformation into α-helix. This conformational change dissociates the TAMRA H-dimer. Many similar transcription factors are involved in oncogenesis, including AP-1.

Rex is a viral protein from human T-cell leukemia virus type I. It functions as a nucleic acid transporter to facilitate appearance of viral RNA in the cytoplasm. The binding between Rex and viral RNA is mediated via the arginine rich motif (ARM) located at the N-terminus of the Rex protein. The ARM is non-structured but upon complexation with the target viral RNA, ARM changes conformation into an S-shaped extended structure. Several other viral RNA-binding proteins function through similar mechanism.

The sequences for GCN4 and Rex beacon designs are:

GCN4-237:
(SEQ ID NO: 26)
CDPAALKRARNTCAARRSRARKLQR MKQLEDK VEELLSK
NYHLENE VARLKKL VGER
GCN4-244:
(SEQ ID NO: 27)
CDPAALKRARNTEAARRSRCRKLQR MKQLEDK VEELLSK
NYHLENE VARLKKL VGER
REX-ARM:
(SEQ ID NO: 28)
MPCTRRRPRRSQRCRPGY

The GCN4 peptides are based on the bZip domain of GCN4 protein (amino acids 226-281). An additional cysteine residue was introduced at its N-terminal end. E237 or A244 are mutated to cysteine for GCN4-237 and GCN4-244, respectively. Both mutations are within the basic region responsible for DNA binding. These GCN4 peptides are expressed as recombinant proteins, fused at the C-terminal end of maltose binding protein. A flexible linker, GGSGG is inserted at the fusion site.

Rex-ARM is modified from the original arginine rich motif of Rex (Rex-ARM), the sequence of which is MPKTRRRPRRSQRKRP (SEQ ID NO:88).

The target and control nuclei acids are as follows.

AP-1 DNA:
(SEQ ID NO: 89)
5′-TTCCTATGACTCATCCAGTT-3′
(SEQ ID NO: 90)
3′-AAGGATACTGAGTAGGTCAA-5′
AT DNA:
(SEQ ID NO: 91)
5′-TTCCTATATATATACCAGTT-3′
(SEQ ID NO: 92)
3′-AAGGATATATATATGGTCAA-5′
Rex aptamer:
(SEQ ID NO: 93)
5′-GGGCGCCGGUACGCAAGUACGACGGUACGCUC-3′
TAR RNA:
(SEQ ID NO: 94)
5′-GGCUCGUGUAGCUCAUUAGCUCCGAGCC-3′

The AP-1 DNA and AT DNA were used for GCN-4 beacons binding assays as the target and control DNAs, respectively. Rex aptamer and TAR RNA were used for Rex beacon binding assays as the target and control RNAs, respectively. For detailed structures of Rex aptamer and TAR RNA, refer to PDB 1EXY and 1MNB, respectively.

Two GCN4 beacons were designed, and one Rex beacon was designed. Cysteine residues were introduced at positions that do not contact target nuclei acids directly. For the GCN4 beacon, S225C/E237C and S225C/A244C were created in the bZIP domain. The former is named GCN4 225/237 beacon and the later is named GCN4 225/244 beacon. For Rex the beacon, K3C/K14C was created. The GCN4 beacons were expressed as a fusion partner at the C-terminal end of MBP.

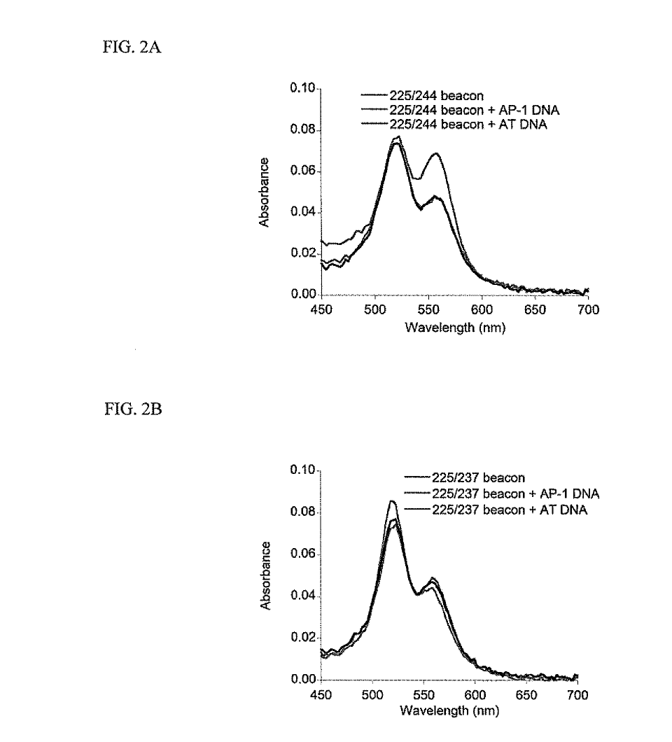

As shown by the shifted absorbance peaks (i.e. shift of highest peak) in FIGS. 2A and 2B, both GCN4 225/237 and GCN4 225/244 beacons allow formation of TAMRA H-dimer. Though, in the presence of a target DNA that contains AP-1 site (AP-1 site is readily recognized by GCN4), GCN4 225/247 beacon exhibits significant alteration of its absorbance spectrum, while the spectrum of GCN4 225/237 beacon is barely changed. The GCN4 225/244 beacon was selected for further characterization.

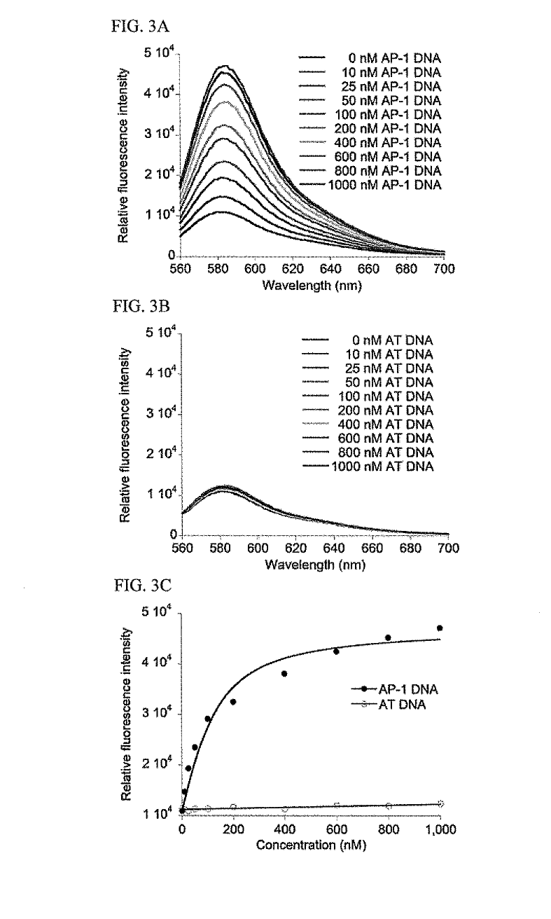

In agreement with the absorbance change, fluorescence emission of GCN4 225/244 beacon is significantly enhanced by presence of AP-1 DNA (FIGS. 3A and 3C). The interaction between GCN4 225/244 beacon and AP-1 is due to specific binding, since the AT DNA does not impose any significant effect (FIGS. 3B and 3C). Titration of 100 nm GCN4 225/247 beacon with increasing concentration of AP-1 results in concentration-dependent fluorescence enhancement, a maxim 4.3-fold fluorescence increase is observed, meanwhile, the AT DNA shows no effects.

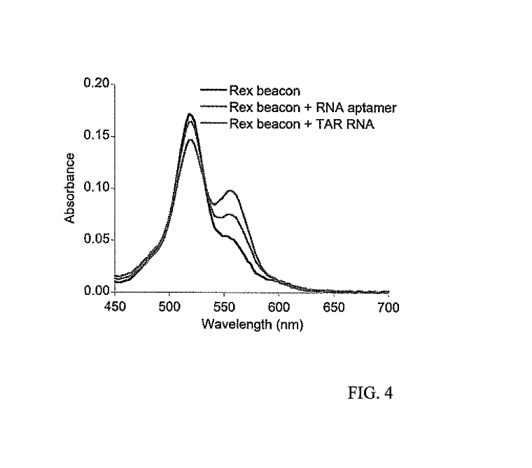

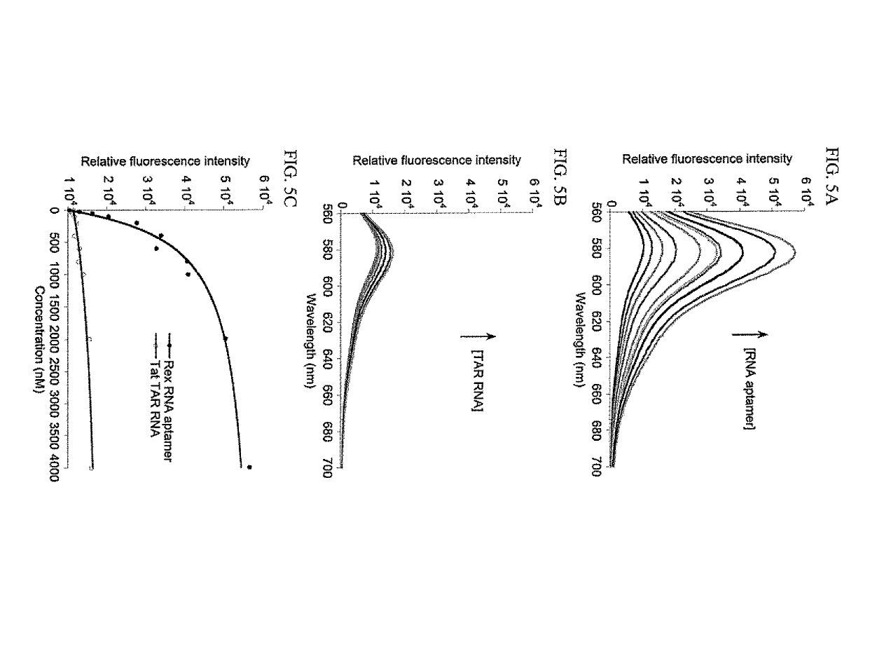

The Rex beacon also shows a shifted absorbance spectra, indicating formation of TAMRA H-dimer. The presence of the RNA aptamer against the ARM of Rex induces a significant change of the beacon's absorbance spectra. Meanwhile, the TAR RNA that has similar structure as compared with the Rex RNA aptamer induces relatively less changes of the absorbance spectra (FIG. 4). The non-specific interaction between Rex beacon and Tat-TAR RNA may come from strong non-specific electrostatic interactions, since the Rex peptide bears a lot arginine residues (7 in total 18 residues in the beacon sequence).

Consistent with the alteration of absorbance spectra, the fluorescence emission of Rex beacon is also significantly enhanced by the Rex RNA aptamer. Titration of 100 nM Rex beacon with the target RNA aptamer shows concentration-dependent increase of fluorescence readout, and a maximum fluorescence enhancement of 4.7-fold is observed. Meanwhile, the Tat TAR RNA induces only 1.4-fold fluorescence enhancement (FIGS. 5A, 5B, and 5C).

Together, the data for GCN4 225/244 beacon and Rex beacon indicate that the H-dimer-based peptide beacons can be used for nucleic acid detection

### Example 3

### DNA Ligase 4 (LigIV) Beacon

XRCC4 is a DNA repair protein. Working together with DNA ligase IV (LigIV), they play fundamental roles in non-homologous end joining (NHEJ). Crystal structure data indicate that, a single LigIV binds asymmetrically to an XRCC4 dimer via a linker peptide (residues 755-782) located between two BRCT domains close to the C-terminal end of LigIV. What is interesting is that, the LigIV linker peptide lacks a hydrophobic core, indicating that it is non-structured in isolation. The binding between the LigIV and XRCC4 is strong enough to withstand high concentration of salt (2 M NaCl) or detergent (7 M urea). The linker region of LigIV represents a good candidate for designing peptide beacons to detect native protein.

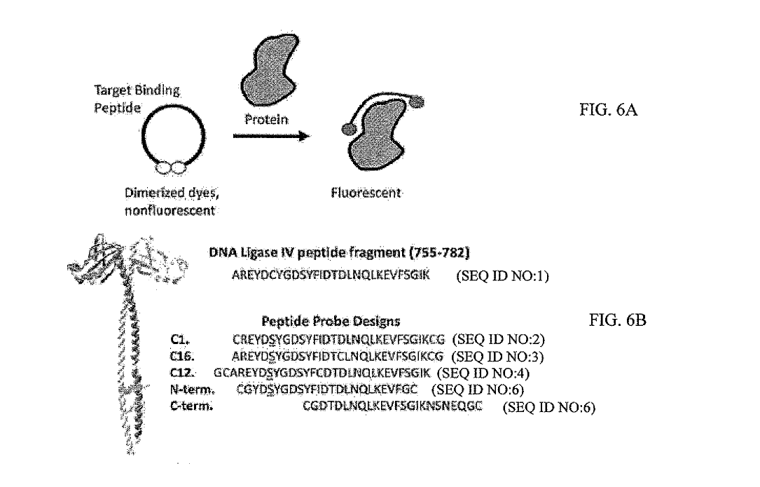

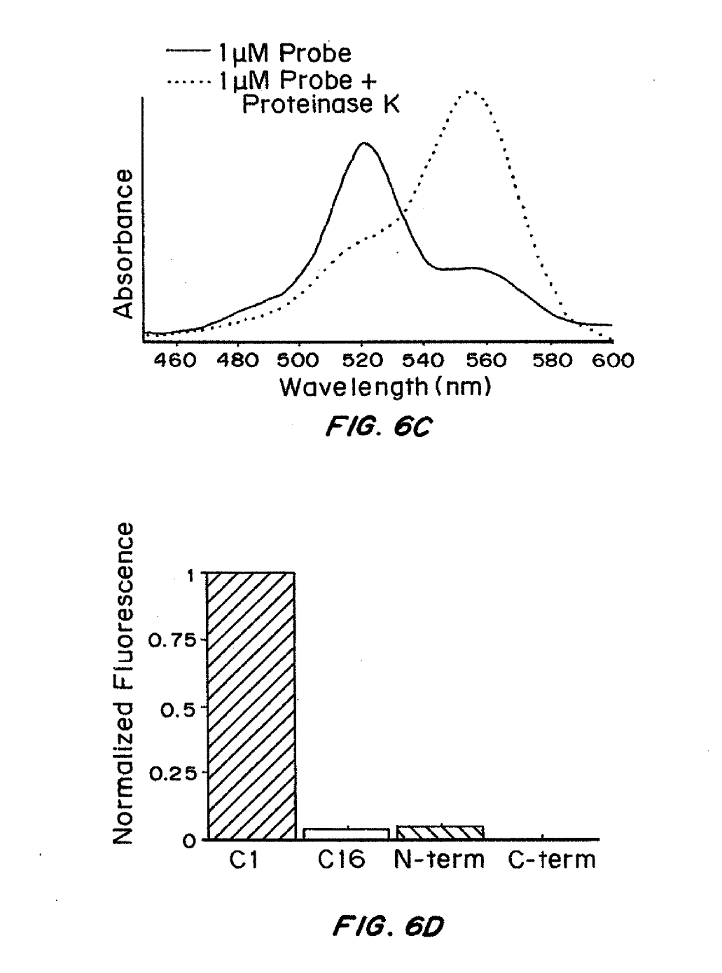

Four peptide beacons were designed, named C1, C16, N-term and C-term. Fluorescence intensities of beacons mixed with XRCC4 were measured to screen the best design. The C1 results in highest fluorescence enhancement (FIG. 6).

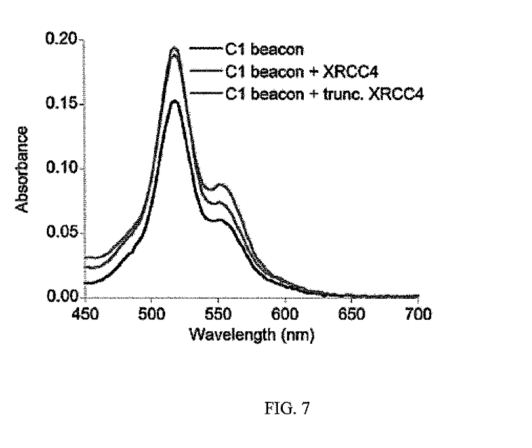

The presence of XRCC4 slightly reverses the major absorbance peak of the C1 beacon, as indicated by slight increase of A550/A520. Meanwhile, truncated XRCC4 (residues 1-155), that does not have the LigIV binding site, even slightly lowers the A550/A520 ratio (FIG. 7). The reason that there is only slight reversing of the major absorbance peak may be due to the fact that there are three tyrosine residues in the C1 peptide, all located between the introduced cysteine residues. The TAMRA fluorophore may interact with the sidechain of tyrosine, which may change its absorbance response to presence of XRCC4. To further verify that C1 beacon does bind XRCC4, gel-shift assays were performed. A clear band is observed for mixture of C1 beacon with XRCC4, while the C1 beacon only sample and mixture of C1 beacon with truncated XRCC4 shows no band (data not shown).

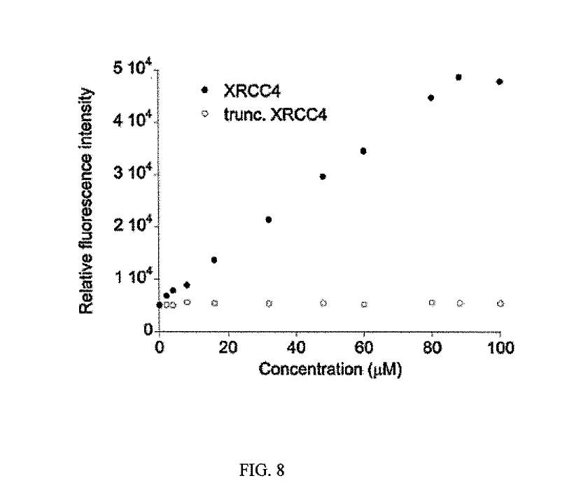

Consistent with the gel-shift assay, significant enhancement of fluorescence emission after addition of XRCC4 protein is observed. As shown in FIG. 8, ˜9.5-fold of fluorescence enhancement is obtained.

C1 beacon was used to test the feasibility of fluorescent imaging of genetically unmodified native protein inside living cells. U2OS cells were transfected with plasmids that express either full length XRCC4 or the truncated XRCC4. Cells were then microinjected with C1 beacon and imaged with a fluorescence microscope. The results are shown in FIG. 8. Only cells transfected with plasmid that express full length XRCC4 exhibit bright TAMRA fluorescence.

### Example 4

### JunLZ-Lb1b4 Beacon

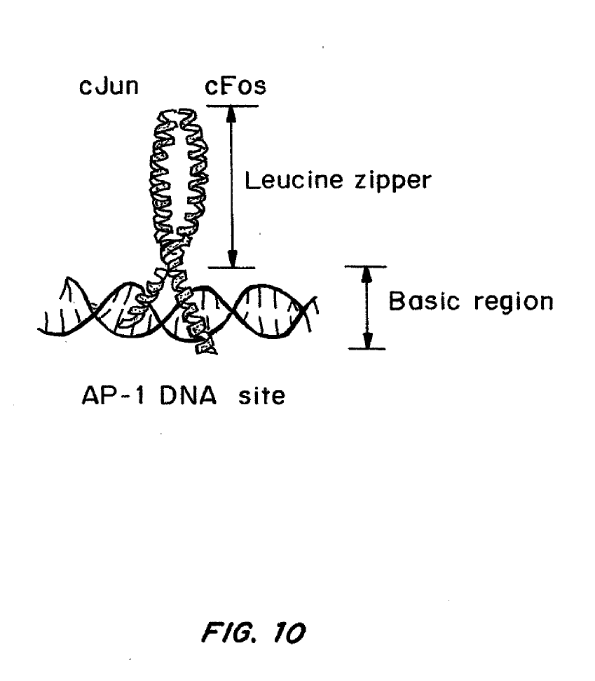

Proteins cJun and cFos are central pieces of the mammalian dimeric transcription factor activator protein-1 (AP-1), which regulates various cellular processes such as differentiation, proliferation, apoptosis and oncogenesis. As illustrated in FIG. 10, both cJun and cFos have a long a helix composed of a region rich in basic residues and a leucine zipper (LZ). The leucine zipper is responsible for dimerization of dun and cFos by forming coiled coil, and the basic region is responsible for DNA binding. This basic region leucine zipper motif is present in many other DNA binding proteins. Together, these proteins constitute the bZIP protein family.

Similar to the K3 and E3 peptides, sequences of leucine zipper are characteristic of heptad repeats, with leucine residue occurring every 7 amino acids. What is different from the artificially designed K3 and E3 peptides is that, native coiled coil-forming peptides often have lower binding affinities due to less optimal residue compositions at a, d, e and g positions. For example, the K_{d} value for binding between the LZs of cFos and cJun at 20° C. is about 700 μM, far lower than the K_{d} value for binding between the K3 and E3 peptides (70 nM). Consequently, to design peptide beacons for detection of native coiled coil-forming peptides, a semirationally designed recognition peptide may be a prerequisite. On the other hand, incorporation of less regular residues at a, d, e and g positions often determines the necessary binding specificity, given that hundreds of coiled coil-forming proteins exist in the confined cellular compartment.

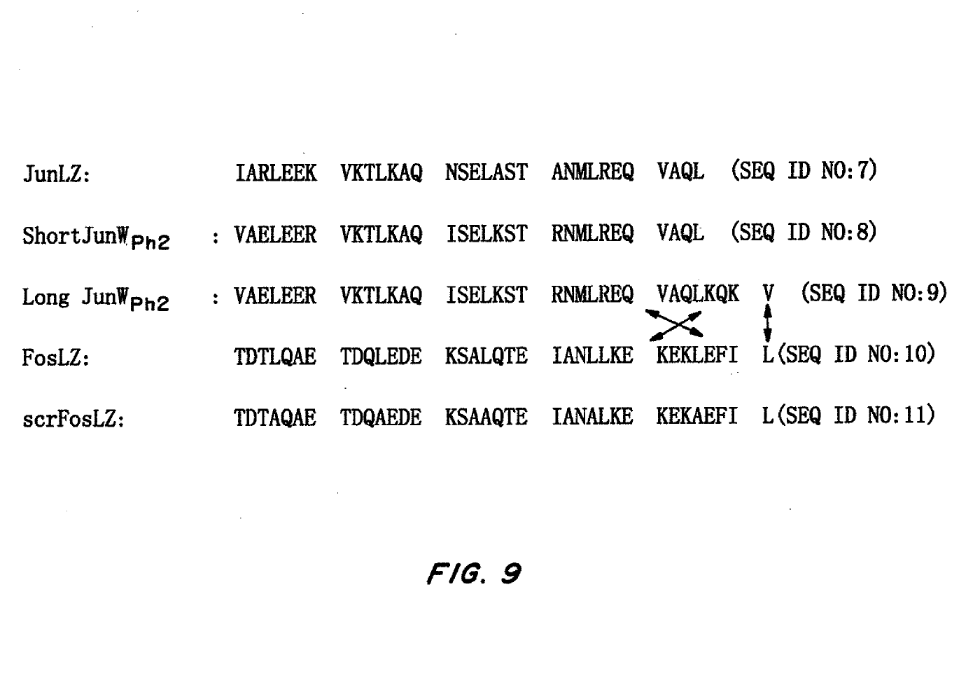

A semirationally evolved variant peptide of the leucine zipper of cJun (JunLZ), named JunWp is selected for beacon design to detect the leucine zipper of cFos (FosLZ). As compared with the wild type JunLZ, JunWₚ has much higher binding affinity (K_{d}˜3 μM) with FosLZ. To further increase the binding affinity, four additional residues right downstream to the leucine zipper of the cJun were also added to the C-terminal end of JunW_{Ph2}. In total, two sets of beacons, the shorter one and the longer one have been designed. Each set contains 4 beacons, with cysteine residues introduced at different positions. Accordingly, the FosLZ is also extended with four residues right downstream to the leucine zipper of cFos. The extended residues are located at e, f, g, a position of heptad repeat, and two more electrostatic interactions and one more hydrophobic interaction is involved, as shown in FIG. 9.

Eight total peptide beacons were designed. A scrambled FosLZ (scrFosLZ) was also designed to serve as the negative control. For scrFosLZ, all the leucine residues at d positions were replaced with alanines. Including the extended FosLZ, all 10 peptides were expressed as recombinant proteins (fused at the C-end of MBP). MBP does not bear any cysteine residue.

JunLZ-Sb1b3:
(SEQ ID NO: 16)
VCELEER VKTLKAQ ICELKST RNMLREQ VAQLA
JunLZ-Sb1b4:
(SEQ ID NO: 18)
VCELEER VKTLKAQ ISELKST RCMLREQ VAQLA
JunLZ-Sb2b5:
(SEQ ID NO: 19)
VAELEER VCTLKAQ ISELKST RNMLREQ VCQLA
JunLZ-Sb3b5:
(SEQ ID NO: 17)
VAELEER VKTLKAQ ICELKST RNMLREQ VCQLA
JunLZ-Lb1b3:
(SEQ ID NO: 20)
VCELEER VKTLKAQ ICELKST RNMLREQ VAQLKQK VA
JunLZ-Lb1b4:
(SEQ ID NO: 22)
VCELEER VKTLKAQ ISELKST RCMLREQ VAQLKQK VA
JunLZ-Lb2b5:
(SEQ ID NO: 23)
VAELEER VCTLKAQ ISELKST RNMLREQ VCQLKQK VA
JunLZ-Lb3b5:
(SEQ ID NO: 21)
VAELEER VKTLKAQ ICELKST RNMLREQ VCQLKQK VA

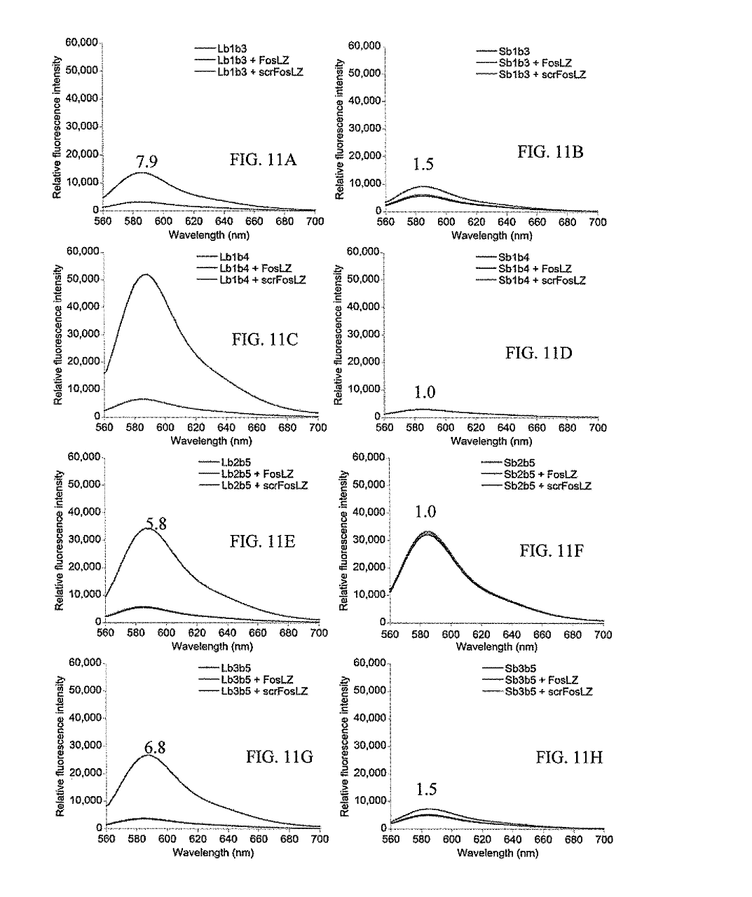

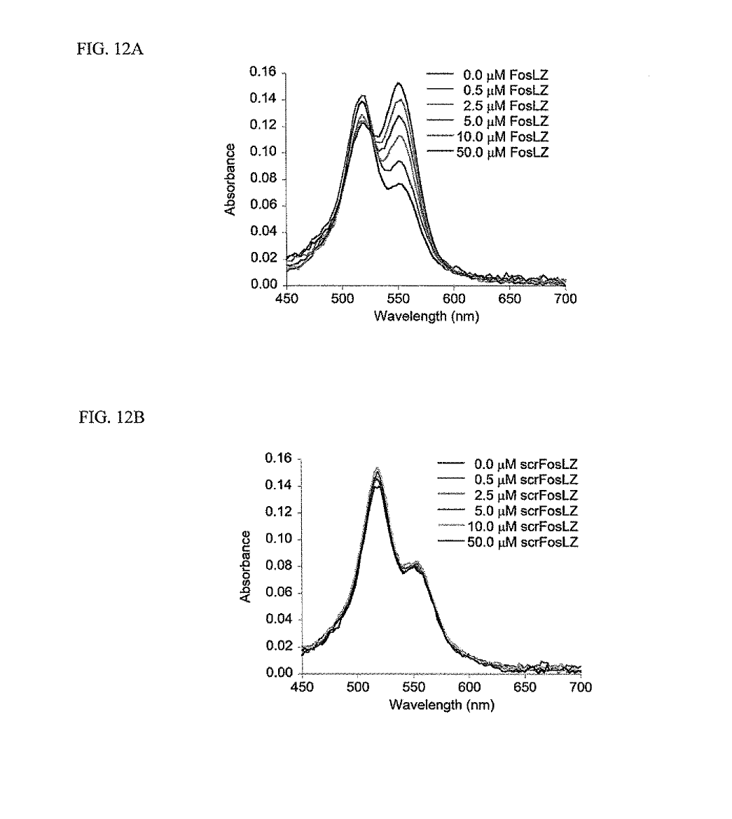

The purified fusion proteins were used directly without removing the MBP fusion partner. To prepare the beacons, TAMRA fluorophores were attached to the fusion proteins via the maleimide chemistry. A fluorescence screening assay indicates that the presence of FosLZ does not induce significant fluorescence enhancement for all the shorter beacon designs. In contrast, all of the longer beacon designs show greatly enhanced fluorescence emission (FIG. 11A-H). The fluorescence enhancement is due to specific coiled coil formation, as scrFosLZ does not induce increased fluorescence intensity (FIG. 11A-H). The fluorescence enhancement of longer beacons can be due to higher binding affinities of these beacons to the FosLZ. Although detailed K_{d} values are not measured, melting temperatures for individual sequences predicted with an online program (bCIPA) indicates that the longer JunWPh2 and FosLZ are more stable than the shorter ones, assuming they both form homodimers (For JunW_{Ph2}, the longer one 60° C., the shorter one 43° C.; For FosLZ, the longer one 12° C., the shorter one 0° C.).

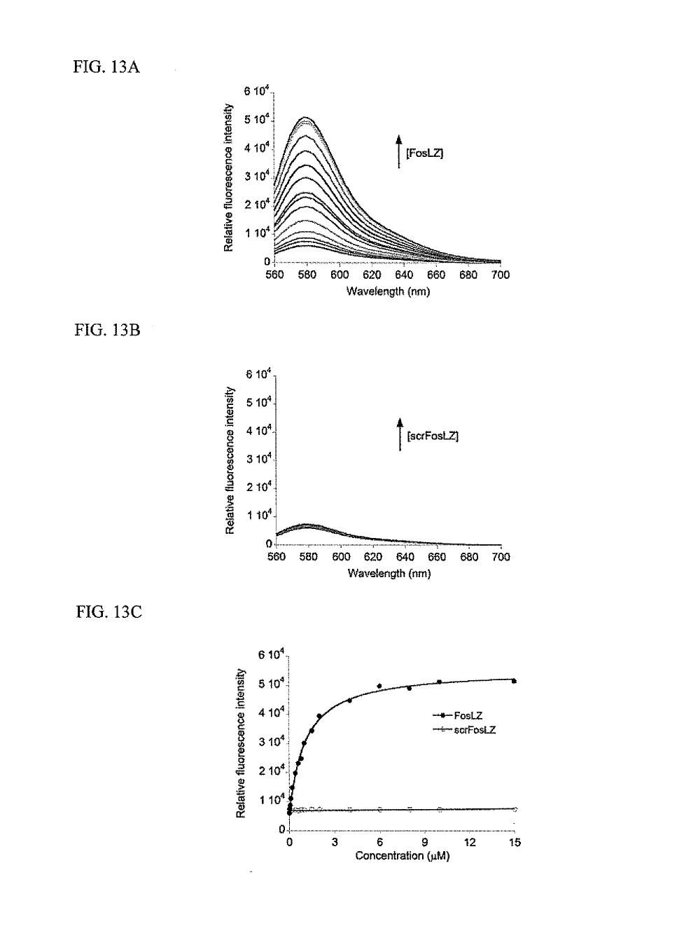

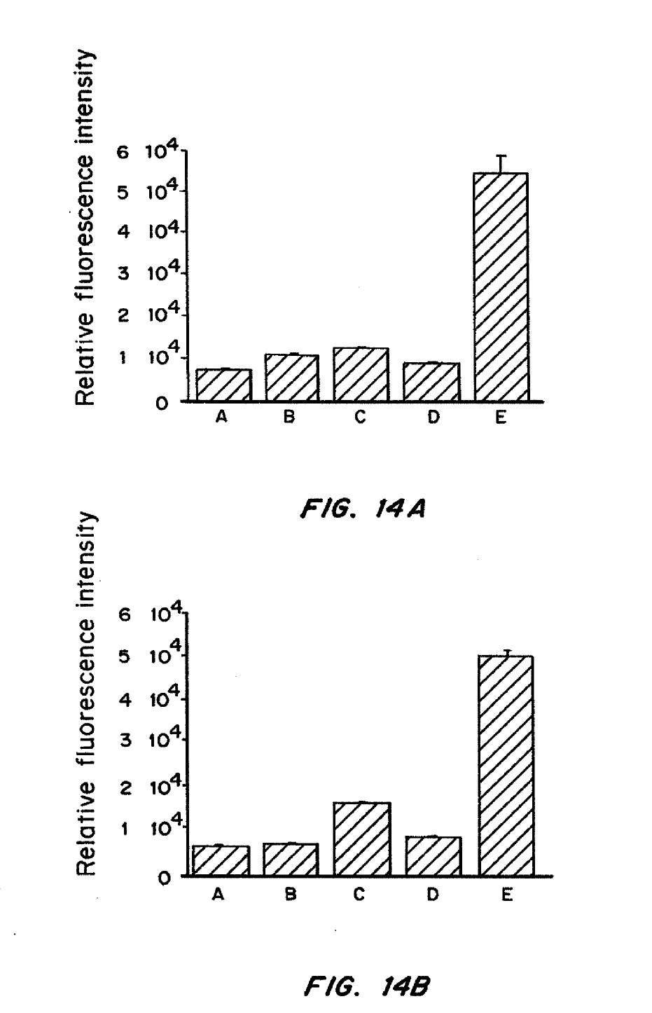

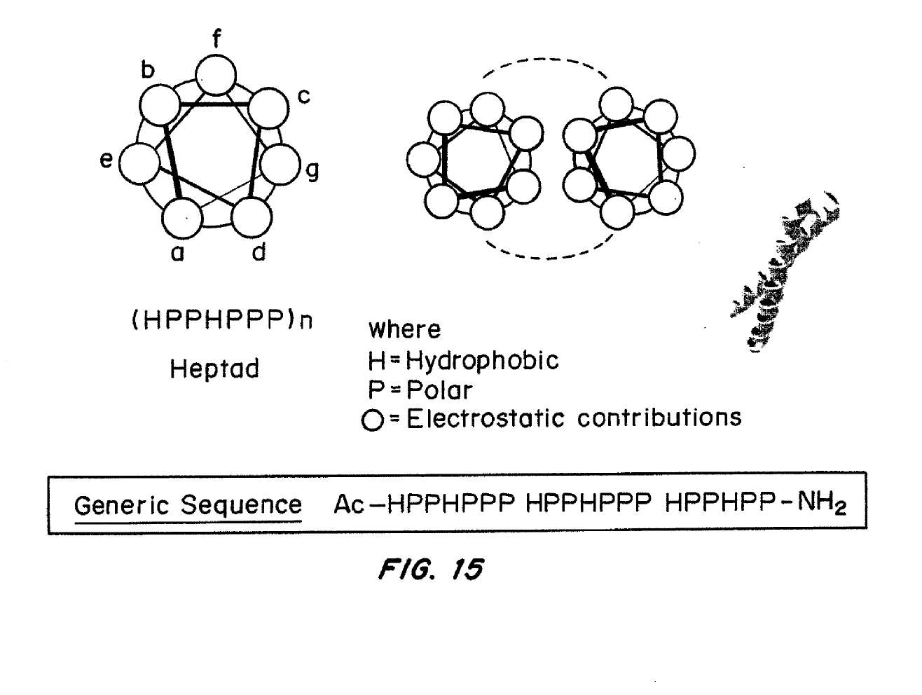

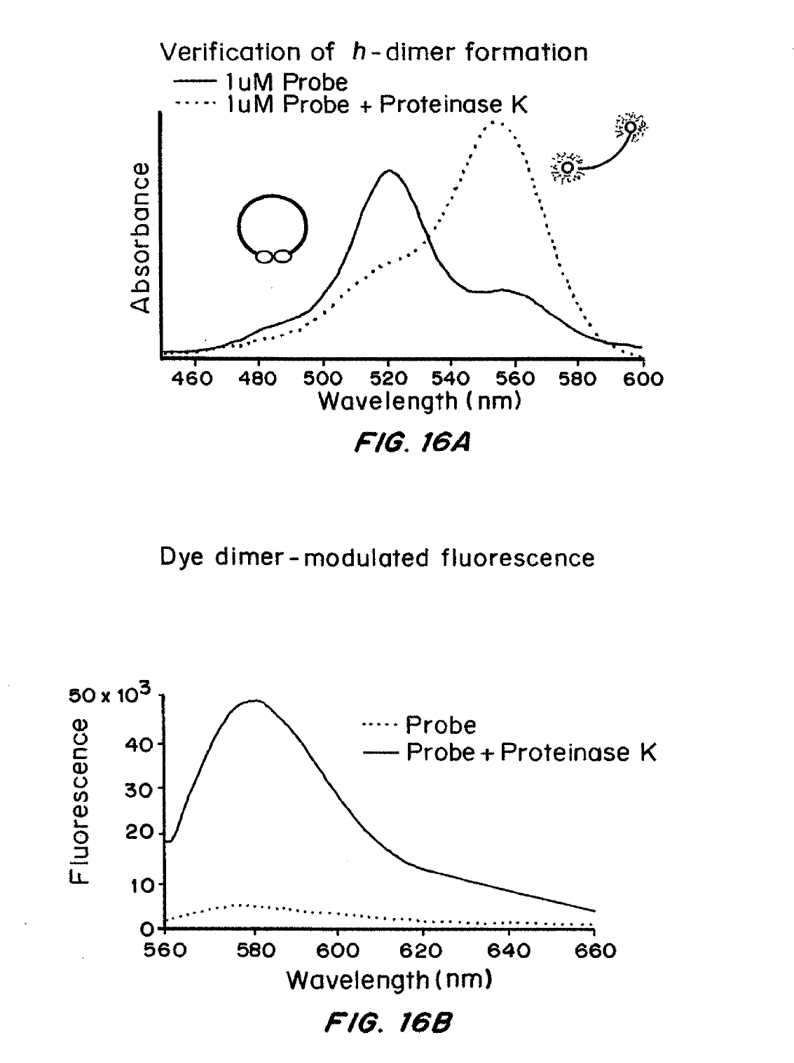

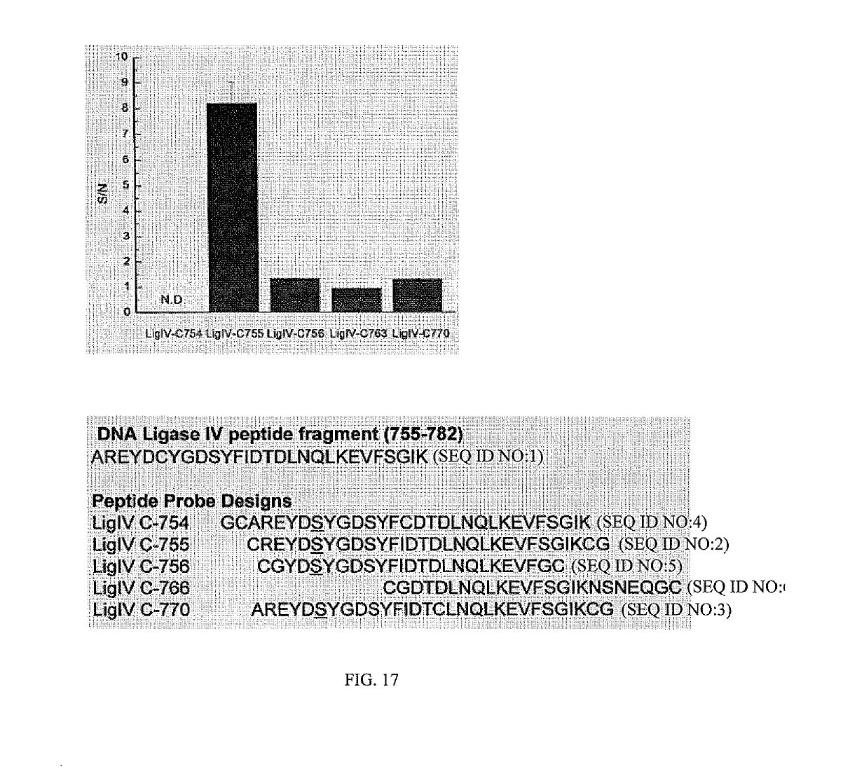

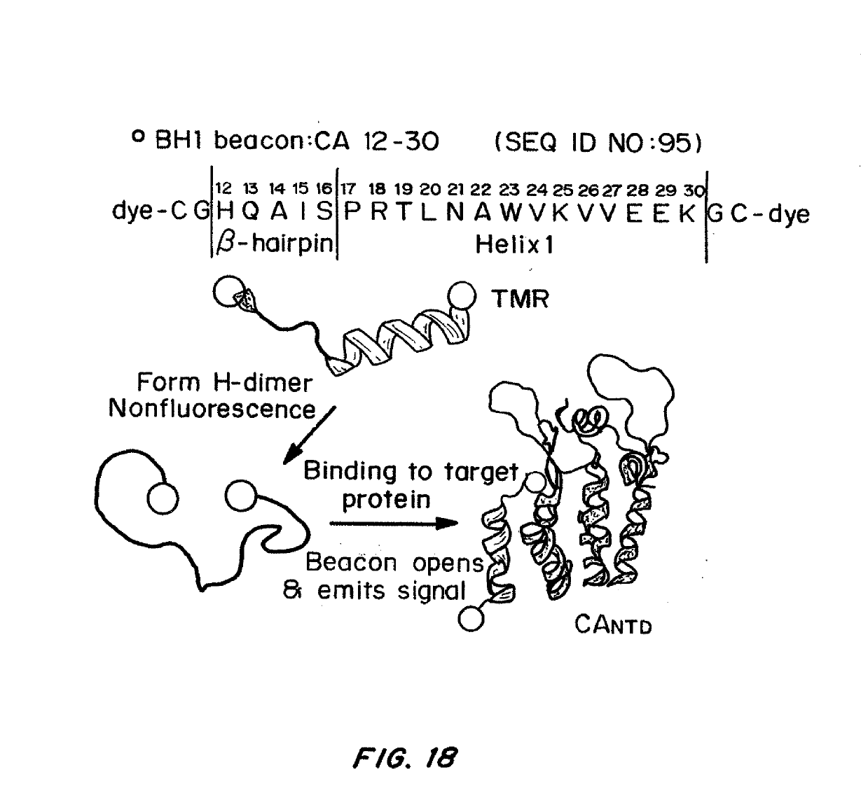

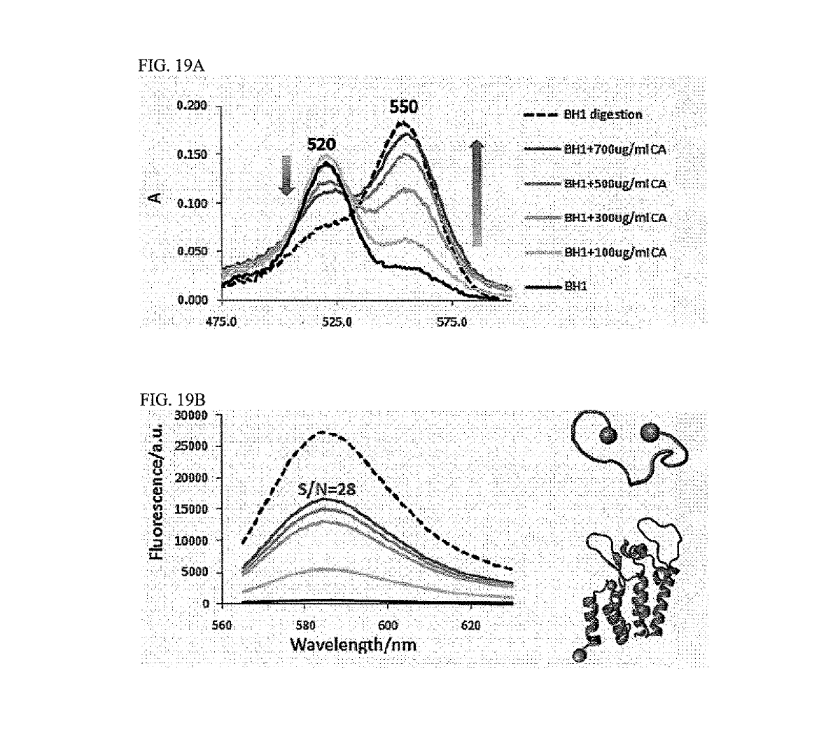

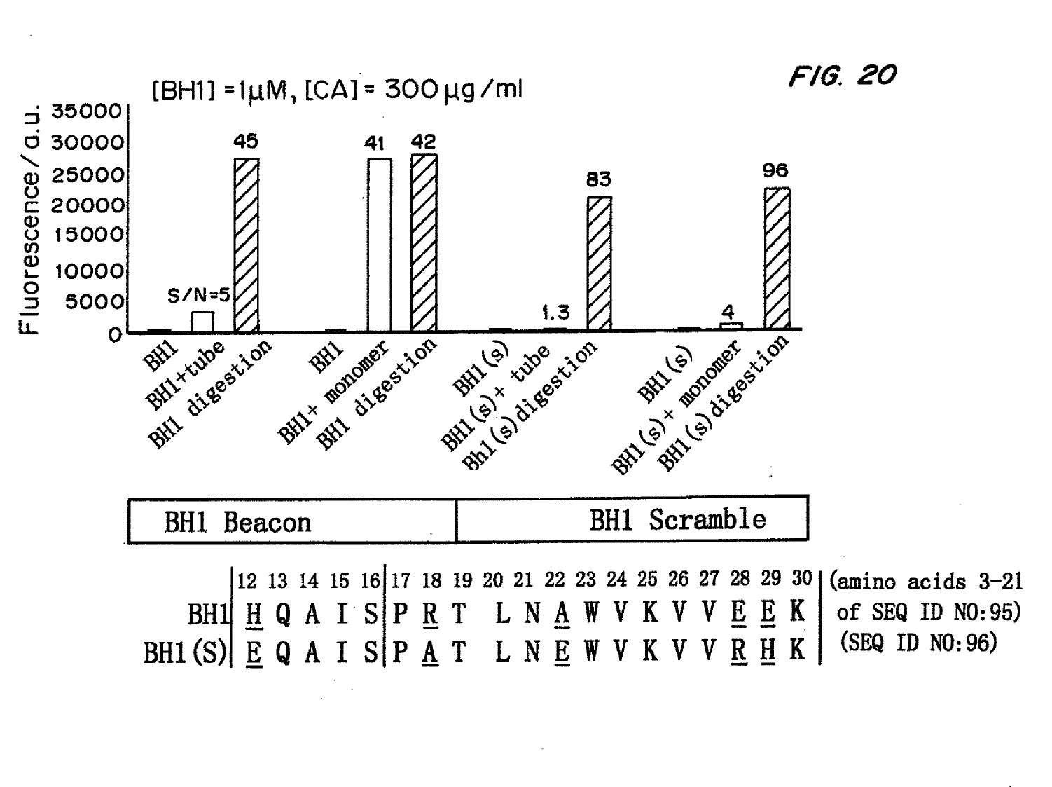

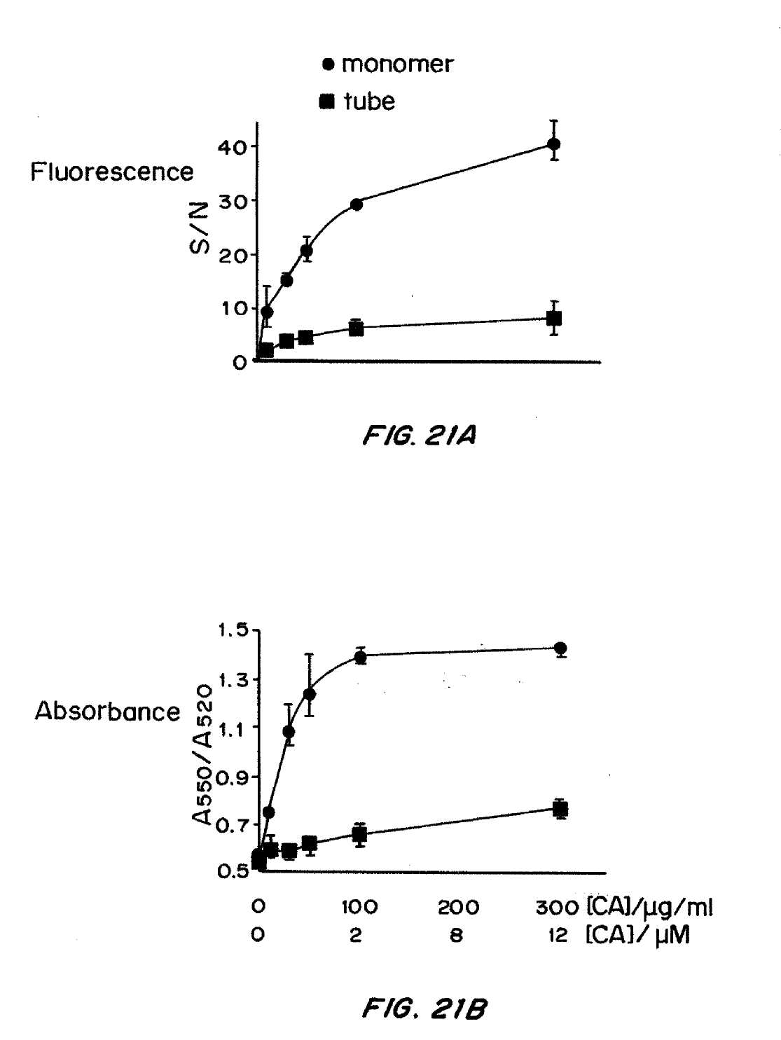

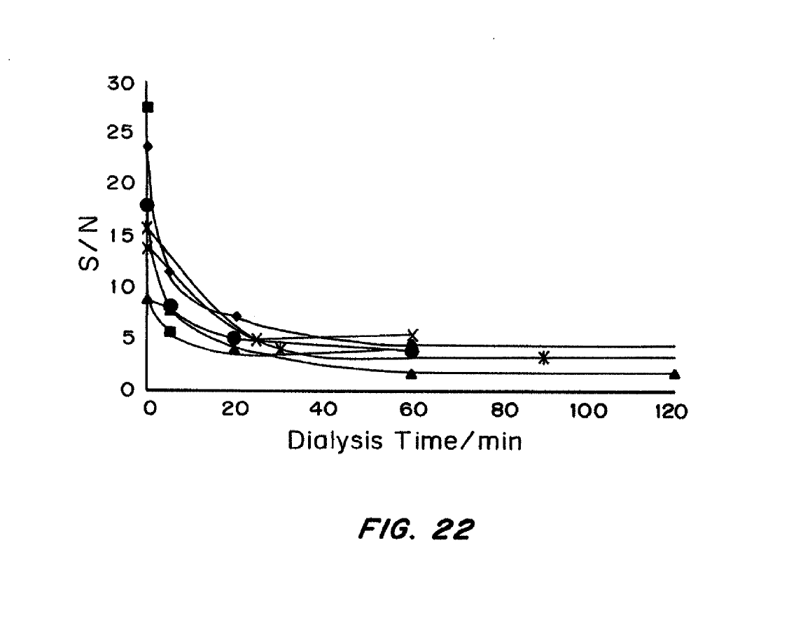

The JunLZ-Lb1b4 beacon shows highest fluorescence enhancement, so it is selected for further characterization. Absorbance spectra of the beacon alone or with the presence of scrFosLZ show typical blue-shifted major absorbance peak (520 nm), while addition of FosLZ to JunLZ-Lb1b4 beacons results in reversing of the major absorbance peak to 550 nm (FIGS. 12A and 12B). Fluorescence emission of JunLZ-Lb1b4 is also significantly enhanced by the presence of FosLZ but not scrFosLZ (FIG. 13A, 13B, 13C), and titration of 100 nM beacon with increasing concentration of FosLZ leads to maxim fluorescence enhancement of ˜8.5-fold is observed.

Both the K3-b1b3 beacon and the JunLZ-Lb1b4 beacon are capable of forming coiled coil by using similar mechanisms. So a concern about the binding specificity naturally arises. To test if there is any significant crosstalk of a beacon with unrelated peptides, the K3-b1b3 beacon and the JunLZ-Lb1b4 beacon were mixed with unrelated peptides and relative fluorescence intensities from different mixtures are compared (FIG. 14). For the K3-b1b3 beacon, no significant non-specific fluorescence enhancement was observed. For the JunLZ-Lb1b4 beacon, E3 induces 2.6-fold fluorescence enhancement. It is not a surprising result, since both peptides have leucine residues at all d positions, and that multiple electrostatic interactions exist between g and g+5 residues. The success of JunLZ-Lb1b4 beacon suggests that, similar beacons can be designed for detection of other native proteins that bear the coiled coil motif, given that a recognition peptide that has high enough binding affinity is identified.

### Example 5

### Polyethylene Glycol Probes

Orthogonal protein-tagging strategies which utilize small molecule probes are important in the quest to understand the mechanics of native protein complexes. To this end, a series of molecular probes were designed which utilize dyes which can form a non-fluorescent ground-state-complex (GSC) to fashion a flexible linker into a traditional molecular beacon.

A series of polyethylene glycol (PEG) probes with varying chain lengths and dye pairs were chemically synthesized and characterized. 5-TAMRA (5-Carboxytetramethylrhodamine) double-labeled probes were shown to form a non-fluorescent GSC in 100 mM PBS. When the dye homodimer is separated, the probes were shown to fluoresce with a signal to noise ratio of approximately 40.

These probes were delivered into the cytoplasm of HeLa cells by Streptolysin O (SLO), after which they diffuse into the nucleus spontaneously. Double-labeled probes displayed very low background signal in their unopened state.

Small, double-labeled PEG molecules which utilize GSC-forming fluorophores are promising fluorogenic probes. They display a high S/N ratio and can easily diffuse into the cell nucleus, making them suitable for labeling proteins in this region. Extensive screening indicates TAMRA and rhodamine green homodimers can form the GSC when conjugated to small PEGs.

### Example 6

### Engineering Protein Beacons for Imaging HIV-1 CA Protein

Peptide beacons for targeting capsid proteins that play an important role in HIV infection were designed. These beacons allowed for detection of capsid proteins.

The capsid proteins form a complex mesh around the viral RNA. Peptide beacons were designed that specifically bind capsid monomers. The BH1 beacon was designed with two fluorescent labels on each end. The peptide backbone contains amino acids 12-30 of the capsid protein with a GC at each end of the backbone (FIG. 18). This peptide backbone contains part of the capsid β-hairpin and the Helix 1. Because capsid proteins interact with each other, the peptide beacon which now resembles a capsid protein can interact with a capsid protein via the helix motif.

As shown in FIG. 19A, BH1 has a shift in absorbance and wavelength in the presence of capsid protein. The absorbance increases as the concentration of capsid protein increases. FIG. 19B shows the increase in fluorescence when the beacon is bound to the capsid protein.

BH1 specifically binds to capsid monomers, not a capsid complex (FIG. 20). As a control, the BH1 beacon was scrambled (BH1(S)). BH1(S) did not bind to capsid complexes or monomers. FIG. 21 further shows the specific binding of Bill to capsid monomers.

Molecular Dynamics (MD) simulation was performed for both BH1 and BH1(S). These studies showed that Helix 1 and 2 of the capsid protein form a hydrophobic groove as a binding site for the peptide beacon. This binding site is only available on capsid monomers. Binding of the peptide beacon was mainly organized by hydrophobic interactions together with electrostatic interactions.

FIG. 22 shows the assembly of capsid proteins into a complex over time.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A fluorogenic peptide probe specific for a target polypeptide, the fluorogenic peptide probe comprising:
a peptide labeled with a fluorogenic label, wherein the fluorogenic peptide probe has an unstructured three dimensional conformation in the absence of the target polypeptide and a structured three dimensional conformation in the presence of the target polypeptide, and wherein the structured three dimensional conformation comprises an alpha-helix that forms a coiled-coil with the target polypeptide allowing generation of a detectable signal from the fluorogenic label in response to an exciting amount of radiation.

2. The fluorogenic peptide probe of claim 1, wherein the fluorogenic peptide probe comprises two fluorogenic labels that form an H-dimer in the unstructured three-dimensional conformation.

3. The fluorogenic peptide probe of claim 1, wherein the fluorogenic label is selected from the group consisting of tetramethylrhodamine and rhodamine green.

4. The fluorogenic peptide probe of claim 1, wherein the target polypeptide is selected from the group consisting of a viral capsid polypeptide, a motor polypeptide, and a DNA-binding transcription factor.

5. A method of detecting a target protein comprising:
contacting the target protein with the fluorogenic peptide probe of claim 1.

6. The method of claim 5, wherein the fluorogenic peptide probe is delivered intracellularly to a cell.

7. The method of claim 5, wherein the detecting occurs in vivo.

8. A method of detecting a viral capsid monomer comprising
contacting the viral capsid monomer with the fluorogenic peptide of claim 1, wherein a detectable signal is produced in response to an exciting amount of radiation when the fluorogenic peptide forms a coiled-coil with the viral capsid monomer.

9. A method of screening for an inhibitor of viral capsid polymerization comprising:
administering a test compound to a cell infected with a virus, wherein the cell comprises the peptide probe of claim 1,
exposing the cell to an exciting amount of radiation, wherein a detectable signal relative to a non-infected cell indicates that the test compound is an inhibitor of viral capsid polymerization.

10. The method of claim 8, wherein the viral capsid monomer is a retrovirus viral capsid monomer.

11. The method of claim 10, wherein the retrovirus is human immunodeficiency virus or hepatitis virus.

12. A method of screening for an inhibitor of intracellular protein complex formation comprising
administering a test compound to a cell comprising at least two proteins that form an intracellular complex, wherein the cell comprises the peptide probe of claim 1,
exposing the cell to an exciting amount of radiation, wherein a detectable signal relative to a control cell indicates that the test compound is an inhibitor of a protein complex formed by the at least two proteins.


SEQUENCE LISTING (96 sequences)

SEQ ID NO: 1 - protein, 28 aa, Artificial Sequence
  DNA IV ligase peptide fragment
AREYDCYGDSYFIDTDLNQLKEVFSGIK

SEQ ID NO: 2 - protein, 30 aa, Artificial Sequence
  DNA Ligase IV probe
CREYDSYGDSYFIDTDLNQLKEVFSGIKCG

SEQ ID NO: 3 - protein, 30 aa, Artificial Sequence
  DNA Ligase IV probe
AREYDSYGDSYFIDTCLNQLKEVFSGIKCG

SEQ ID NO: 4 - protein, 30 aa, Artificial Sequence
  DNA Ligase IV probe
GCAREYDSYGDSYFCDTDLNQLKEVFSGIK

SEQ ID NO: 5 - protein, 25 aa, Artificial Sequence
  DNA Ligase IV probe
CGYDSYGDSYFIDTDLNQLKEVFGC

SEQ ID NO: 6 - protein, 24 aa, Artificial Sequence
  DNA Ligase IV probe
CGDTDLNQLKEVFSGIKNSNEQGC

SEQ ID NO: 7 - protein, 32 aa, Artificial Sequence
  Jun probe
IARLEEKVKTLKAQNSELASTANMLREQVAQL

SEQ ID NO: 8 - protein, 32 aa, Artificial Sequence
  Jun probe
VAELEERVKTLKAQISELKSTRNMLREQVAQL

SEQ ID NO: 9 - protein, 36 aa, Artificial Sequence
  Jun probe
VAELEERVKTLKAQISELKSTRNMLREQVAQLKQKV

SEQ ID NO: 10 - protein, 36 aa, Artificial Sequence
  Fos probe
TDTLQAETDQLEDEKSALQTEIANLLKEKEKLEFIL

SEQ ID NO: 11 - protein, 36 aa, Artificial Sequence
  scrambled Fos probe
TDTAQAETDQAEDEKSAAQTEIANALKEKEKAEFIL

SEQ ID NO: 12 - protein, 23 aa, Artificial Sequence
  probe
KICALKAKIAALKAKICALKAGY

SEQ ID NO: 13 - protein, 23 aa, Artificial Sequence
  probe
KICALKAKIAALKAKIAALKAGY

SEQ ID NO: 14 - protein, 23 aa, Artificial Sequence
  probe or target sequence for a probe
EIAALEKEIAALEKEIAALEKGY

SEQ ID NO: 15 - protein, 23 aa, Artificial Sequence
  scrambled probe or target sequence for probe
EAAAAEKEAAAAEKEAAAAEKGY

SEQ ID NO: 16 - protein, 33 aa, Artificial Sequence
  Jun probe
VCELEERVKTLKAQICELKSTRNMLREQVAQLA

SEQ ID NO: 17 - protein, 33 aa, Artificial Sequence
  Jun probe
VAELEERVKTLKAQICELKSTRNMLREQVCQLA

SEQ ID NO: 18 - protein, 33 aa, Artificial Sequence
  Jun probe
VCELEERVKTLKAQISELKSTRCMLREQVAQLA

SEQ ID NO: 19 - protein, 33 aa, Artificial Sequence
  Jun probe
VAELEERVCTLKAQISELKSTRNMLREQVCQLA

SEQ ID NO: 20 - protein, 37 aa, Artificial Sequence
  Jun probe
VCELEERVKTLKAQICELKSTRNMLREQVAQLKQKVA

SEQ ID NO: 21 - protein, 37 aa, Artificial Sequence
  Jun probe
VAELEERVKTLKAQICELKSTRNMLREQVCQLKQKVA

SEQ ID NO: 22 - protein, 37 aa, Artificial Sequence
  Jun probe
VCELEERVKTLKAQISELKSTRCMLREQVAQLKQKVA

SEQ ID NO: 23 - protein, 37 aa, Artificial Sequence
  Jun probe
VAELEERVCTLKAQISELKSTRNMLREQVCQLKQKVA

SEQ ID NO: 24 - protein, 37 aa, Artificial Sequence
  Fos probe
TDTLQAETDQLEDEKSALQTEIANLLKEKEKLEFILA

SEQ ID NO: 25 - protein, 37 aa, Artificial Sequence
  scrambled Fos probe
TDTAQAETDQAEDEKSAAQTEIANALKEKEKAEFILA

SEQ ID NO: 26 - protein, 57 aa, Artificial Sequence
  GCN4 probe
CDPAALKRARNTCAARRSRARKLQRMKQLEDKVEELLSKNYHLENEVARLKKLVGER

SEQ ID NO: 27 - protein, 57 aa, Artificial Sequence
  GCN4 probe
CDPAALKRARNTEAARRSRCRKLQRMKQLEDKVEELLSKNYHLENEVARLKKLVGER

SEQ ID NO: 28 - protein, 18 aa, Artificial Sequence
  Rex - arginine rich motif probe
MPCTRRRPRRSQRCRPGY

SEQ ID NO: 29 - protein, 21 aa, Artificial Sequence
  probe
KIAALKEKIAALKEKIAALKE

SEQ ID NO: 30 - protein, 16 aa, Artificial Sequence
  Rex-arginine rich motif probe
MPKTRRRPRRSQRKRP

SEQ ID NO: 31 - DNA, 90 bp, Artificial Sequence
  coding sequence of E3 peptide
gatccggaggtagtggtggagagatcgctgcactggagaaagaaattgcagctctcgaaaaggagatcgctgctctggagaagccataag

SEQ ID NO: 32 - DNA, 90 bp, Artificial Sequence
  coding sequence for E3 peptide
tcgacttatggcttctccagagcagcgatctccttttcgagagctgcaatttctttctccagtgcagcgatctctccaccactacctccg

SEQ ID NO: 33 - DNA, 42 bp, Artificial Sequence
  primer for maltose binding protein
gttactggatccaaaatcgaagaaggtaaactggtaatctgg

SEQ ID NO: 34 - DNA, 36 bp, Artificial Sequence
  primer for maltose binding protein
gttactgtcgacagtctgcgcgtctttcagggcttc

SEQ ID NO: 35 - DNA, 46 bp, Artificial Sequence
  oligonucleotide
tcgacggcggcagcggcggcgtgtgcgaactggaagaacgcgtgaa

SEQ ID NO: 36 - DNA, 46 bp, Artificial Sequence
  oligonucleotide
ggttttcacgcgttcttccagttcgcacacgccgccgctgccgccg

SEQ ID NO: 37 - DNA, 46 bp, Artificial Sequence
  oligonucleotide
tcgacggcggcagcggcggcgtggcagaactggaagaacgcgtgaa

SEQ ID NO: 38 - DNA, 46 bp, Artificial Sequence
  oligonucleotide
ggttttcacgcgttcttccagttctgccacgccgccgctgccgccg

SEQ ID NO: 39 - DNA, 46 bp, Artificial Sequence
  oligonucleotide
tcgacggcggcagcggcggcgtggcagaactggaagaacgcgtgtg

SEQ ID NO: 40 - DNA, 46 bp, Artificial Sequence
  oligonucleotide
ggtgcacacgcgttcttccagttctgccacgccgccgctgccgccg

SEQ ID NO: 41 - DNA, 47 bp, Artificial Sequence
  oligonucleotide
aaccctgaaagcgcagatttgcgaactgaaaagcacccgcaacatgc

SEQ ID NO: 42 - DNA, 47 bp, Artificial Sequence
  oligonucleotide
cgcagcatgttgcgggtgcttttcagttcgcaaatctgcgctttcag

SEQ ID NO: 43 - DNA, 47 bp, Artificial Sequence
  oligonucleotide
aaccctgaaagcgcagattagcgaactgaaaagcacccgctgcatgc

SEQ ID NO: 44 - DNA, 47 bp, Artificial Sequence
  oligonucleotide
cgcagcatgcagcgggtgcttttcagttcgctaatctgcgctttcag

SEQ ID NO: 45 - DNA, 47 bp, Artificial Sequence
  oligonucleotide
caccctgaaagcgcagattagcgaactgaaaagcacccgcaacatgc

SEQ ID NO: 46 - DNA, 47 bp, Artificial Sequence
  oligonucleotide
cgcagcatgttgcgggtgcttttcagttcgctaatctgcgctttcag

SEQ ID NO: 47 - DNA, 34 bp, Artificial Sequence
  oligonucleotide
tgcgcgaacaggtgtgccagctggcgtaactgca

SEQ ID NO: 48 - DNA, 26 bp, Artificial Sequence
  oligonucleotide
gttacgccagctggcacacctgttcg

SEQ ID NO: 49 - DNA, 34 bp, Artificial Sequence
  oligonucleotide
tgcgcgaacaggtggcacagctggcgtaactgca

SEQ ID NO: 50 - DNA, 26 bp, Artificial Sequence
  oligonucleotide
gttacgccagctgtgccacctgttcg

SEQ ID NO: 51 - DNA, 46 bp, Artificial Sequence
  oligonucleotide
tgcgcgaacaggtgtgccagctgaaacagaaagtggcgtaactgca

SEQ ID NO: 52 - DNA, 38 bp, Artificial Sequence
  oligonucleotide
gttacgccactttctgtttcagctggcacacctgttcg

SEQ ID NO: 53 - DNA, 46 bp, Artificial Sequence
  oligonucleotide
tgcgcgaacaggtggcacagctgaaacagaaagtggcgtaactgca

SEQ ID NO: 54 - DNA, 38 bp, Artificial Sequence
  oligonucleotide
gttacgccactttctgtttcagctgtgccacctgttcg

SEQ ID NO: 55 - DNA, 44 bp, Artificial Sequence
  oligonucleotide
tcgacggcggcagcggcggcaccgataccctgcaagcggaaacc

SEQ ID NO: 56 - DNA, 44 bp, Artificial Sequence
  oligonucleotide
gatcggtttccgcttgcagggtatcggtgccgccgctgccgccg

SEQ ID NO: 57 - DNA, 47 bp, Artificial Sequence
  oligonucleotide
gatcagctggaagatgaaaaaagcgcgctgcaaaccgaaattgcgaa

SEQ ID NO: 58 - DNA, 47 bp, Artificial Sequence
  oligonucleotide
caggttcgcaatttcggtttgcagcgcgcttttttcatcttccagct

SEQ ID NO: 59 - DNA, 48 bp, Artificial Sequence
  oligonucleotide
cctgctgaaagaaaaagaaaaactggaatttattctggcgtaactgca

SEQ ID NO: 60 - DNA, 40 bp, Artificial Sequence
  oligonucleotide
gttacgccagaataaattccagtttttctttttctttcag

SEQ ID NO: 61 - DNA, 44 bp, Artificial Sequence
  oligonucleotide
tcgacggcggcagcggcggcaccgataccgcgcaggcggaaacc

SEQ ID NO: 62 - DNA, 44 bp, Artificial Sequence
  oligonucleotide
gatcggtttccgcctgcgcggtatcggtgccgccgctgccgccg

SEQ ID NO: 63 - DNA, 45 bp, Artificial Sequence
  oligonucleotide
gatcaggcggaggatgaaaaaagcgcggcgcagaccgaaattgcg

SEQ ID NO: 64 - DNA, 45 bp, Artificial Sequence
  oligonucleotide
cgttcgcaatttcggtctgcgccgcgcttttttcatcctccgcct

SEQ ID NO: 65 - DNA, 50 bp, Artificial Sequence
  oligonucleotide
aacgcgctgaaagaaaaagaaaaagcggaatttattctggcgtaactgca

SEQ ID NO: 66 - DNA, 42 bp, Artificial Sequence
  oligonucleotide
gttacgccagaataaattccgctttttctttttctttcagcg

SEQ ID NO: 67 - DNA, 48 bp, Artificial Sequence
  oligonucleotide
tcgacggcggcagcggcggctgcgatccggcggcgctgaaacgcgcgc

SEQ ID NO: 68 - DNA, 48 bp, Artificial Sequence
  oligonucleotide
ttgcgcgcgcgtttcagcgccgccggatcgcagccgccgctgccgccg

SEQ ID NO: 69 - DNA, 46 bp, Artificial Sequence
  oligonucleotide
gcaacacctgcgcggcgcgccgcagccgcgcgcgcaaactgcaacg

SEQ ID NO: 70 - DNA, 46 bp, Artificial Sequence
  oligonucleotide
catgcgttgcagtttgcgcgcgcggctgcggcgcgccgcgcaggtg

SEQ ID NO: 71 - DNA, 46 bp, Artificial Sequence
  oligonucleotide
gcaacaccgaagcggcgcgccgcagccgctgccgcaaactgcaacg

SEQ ID NO: 72 - DNA, 46 bp, Artificial Sequence
  oligonucleotide
catgcgttgcagtttgcggcagcggctgcggcgcgccgcttcggtg

SEQ ID NO: 73 - DNA, 50 bp, Artificial Sequence
  oligonucleotide
catgaaacagctggaagataaagtggaagaactgctgagcaaaaactatc

SEQ ID NO: 74 - DNA, 50 bp, Artificial Sequence
  oligonucleotide
agatgatagtttttgctcagcagttcttccactttatcttccagctgttt

SEQ ID NO: 75 - DNA, 55 bp, Artificial Sequence
  oligonucleotide
atctggaaaacgaagtggcgcgcctgaaaaaactggtgggcgaacgctaactgca

SEQ ID NO: 76 - DNA, 47 bp, Artificial Sequence
  oligonucleotide
gttagcgttcgcccaccagttttttcaggcgcgccacttcgttttcc

SEQ ID NO: 77 - DNA, 44 bp, Artificial Sequence
  primer
gttattggatccgagagaaaaataagcagaatccaccttgtttc

SEQ ID NO: 78 - DNA, 43 bp, Artificial Sequence
  primer
gttattgtcgacttaaatctcatcaaagaggtcttctgggctg

SEQ ID NO: 79 - DNA, 47 bp, Artificial Sequence
  primer
cgtagtactggatccgagagaaaaataagcagaatccaccttgtttc

SEQ ID NO: 80 - DNA, 46 bp, Artificial Sequence
  primer
tgcatgagctccctttcattttctttctgcaggtgctcatttttgg

SEQ ID NO: 81 - DNA, 54 bp, Artificial Sequence
  primer
gccctgggatccgccaccatggagagaaaaataagcagaatccaccttgtttct

SEQ ID NO: 82 - DNA, 44 bp, Artificial Sequence
  primer
gccatgtctagattaaatctcatcaaagaggtcttctgggctgc

SEQ ID NO: 83 - DNA, 54 bp, Artificial Sequence; identical to SEQ ID NO: 81
  primer

SEQ ID NO: 84 - DNA, 77 bp, Artificial Sequence
  primer
tgcattctagactacctttcattttctttctgcaggtgctcatttttggcttgattttctgcaatggtgtccaagca

SEQ ID NO: 85 - protein, 24 aa, Artificial Sequence
  probe
KICALKEKICALKEKIAALKEGWP

SEQ ID NO: 86 - protein, 24 aa, Artificial Sequence
  probe
KICALKEKIAALKEKICALKEGWP

SEQ ID NO: 87 - protein, 24 aa, Artificial Sequence
  probe
EIAALEKEIAALEKEIAALEKGWP

SEQ ID NO: 88 - protein, 16 aa, Artificial Sequence
  Rex-arginine rich motif probe
MPKTRRRPRRSQRKRP

SEQ ID NO: 89 - DNA, 20 bp, Artificial Sequence
  AP-1 probe
ttcctatgactcatccagtt

SEQ ID NO: 90 - DNA, 20 bp, Artificial Sequence
  AP-1 probe
aaggatactgagtaggtcaa

SEQ ID NO: 91 - DNA, 20 bp, Artificial Sequence
  AT probe
ttcctatatatataccagtt

SEQ ID NO: 92 - DNA, 20 bp, Artificial Sequence
  AT probe
aaggatatatatatggtcaa

SEQ ID NO: 93 - RNA, 32 nt, Artificial Sequence
  Rex aptamer probe
gggcgccgguacgcaaguacgacgguacgcuc

SEQ ID NO: 94 - RNA, 28 nt, Artificial Sequence
  Rex aptamer probe
ggcucguguagcucauuagcuccgagcc

SEQ ID NO: 95 - protein, 23 aa, Artificial Sequence
  Probe
CGHQAISPRTLNAWVKVVEEKGC

SEQ ID NO: 96 - protein, 19 aa, Artificial Sequence
  scrambled probe
EQAISPATLNEWVKVVRHK